(19) United States
(12) Patent Application Publication
(10) Pub. No.: US 2025/0074932 A1
(43) Pub. Date: Mar. 6, 2025

(54) CATALYTIC STEREOSELECTIVE GLYCOSYLATION

(71) Applicant: Purdue Research Foundation, West Lafayette, IN (US)

(72) Inventors: Abram Joseph Axelrod, West Lafayette, IN (US); Charles Enlow, Lafayette, IN (US); Bhaswati Ghosh, Brighton, MA (US)

(21) Appl. No.: 18/792,824

(22) Filed: Aug. 2, 2024

Related U.S. Application Data
(60) Provisional application No. 63/536,124, filed on Sep. 1, 2023.

(51) Int. Cl. C07H 17/04; B01J 31/02; C07H 15/04; C07H 15/203

(52) U.S. Cl. CPC C07H 17/04; B01J 31/0271; C07H 15/04; C07H 15/203

(57) ABSTRACT

A catalytic stereoselective glycosylation method for preparing an α-1,2-cis glycosidic linkage, which method comprises reacting a glycosyl donor with a glycosyl acceptor in the presence of an isothiourea catalyst, a base, and a solvent.

### CROSS-REFERENCE TO RELATED APPLICATIONS

This application claims priority to U.S. provisional patent application No. 63/536,124, which was filed Sep. 1, 2023, and which is hereby incorporated by reference in its entirety.

### TECHNICAL FIELD

The present disclosure relates to a method for catalytic stereoselective glycosylation for preparing an α-1,2-cis glycosidic linkage using an isothiourea catalyst.

### BACKGROUND

Oligosaccharides are found in various biologically relevant therapeutics and medicinal agents and constitute the key motif of numerous biologics and therapeutics. For oligosaccharides, chemical synthesis is a more practical pathway to obtain these molecules in single isoform, homogeneous species. But, compared to peptides or nucleic acids, the chemical synthesis of oligosaccharides in both high yield and stereoselectivity is still challenging.

Exerting stereocontrol in glycosylation is a fundamental goal within carbohydrate synthesis and is of the utmost importance for the efficient preparation of single-isoform oligosaccharides. This is especially prescient in glycobiology and medicine, where defined, homogenous glycan constructs are required for precise interrogation. The preparation of complex carbohydrate structures is not always straightforward, considering the numerous variables inherent to glycosidic bond formation. For example, while the synthesis of trans glycosidic bonds, both beta-1, 2-trans or alpha-1, 2-trans, can be easily achieved with anchimeric assistance through protecting groups, the analogous preparation of alpha-1,2-cis glycosides is less predictable.

Numerous specialized methods have been developed to achieve control of anomeric selectivity during glycosylation. But this can be challenging due to the intricacies stemming from both donor and acceptor structures, leading to competing S_{N}1 and S_{N}2 pathways and resulting in a loss of selectivity and efficiency, due to stereoelectronic nuances promoting a fluid S_{N}1 and S_{N}2 continuum, eroding selectivity. A number of methodologies have been advanced to address these challenges, including auxiliaries or directing groups and exogenous modulators. These strategies operate through the stoichiometric activation of a leaving group to generate an oxocarbenium, which is then engaged by the auxiliary or additive to template glycosylation.

Hence, there is an unmet need for a method for glycosylation that controls anomeric selectivity during glycosylation to provide α-1,2-cis glycosidic linkages in both excellent yield and stereoselectivity. It is an object of the present disclosure to provide such a method. This and other objects and advantages, as well as inventive features, will be apparent from the detailed description.

### SUMMARY

Provided is a catalytic stereoselective glycosylation method for preparing an α-1,2-cis glycosidic linkage, which method comprises:
reacting a glycosyl donor of formula (I):

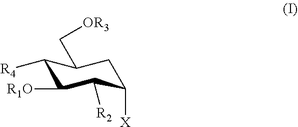

wherein
each of R₁ and R₃ is independently a substituted or an unsubstituted group selected from alkyl, heteroalkyl, cycloalkyl, heterocycloalkyl, aryl, heteroaryl, alkynyl, alkenyl, heteroarylalkyl, heteroalkylaryl, arylalkenyl, allyl, arylalkyl, alkylcarboxyl, arylcarboxyl, trialkylsilyl, diarylalkylsilyl, dialkylarylsilyl, alkoxyalkyl, tetrahydropyranyl, benzyl, and benzoyl; wherein each substituted group can be independently substituted with at least one of alkyl, aryl, heteroaryl, halo, cyano, alkoxy, amino, carboxyl, amide, and ester;
each of R₂ and R₄ is independently alkyl, N₃ or OR₁, wherein R₁ is as defined above; or
any two adjacent groups, such as OR₁ and R₂, OR₁ and R₄, and OR₃ and R₄ are linked together to form a 5- to 7-membered cyclic ring, wherein the cyclic ring can be optionally independently substituted with at least one of alkyl, aryl, heteroaryl, halo, cyano, alkoxy, trialkylsilyl, diarylalkylsilyl, amino, carboxyl, amide, and ester group; and
X is a halide selected from Cl, Br, I and F;
with a glycosyl acceptor in the presence of a base and an isothiourea catalyst of formula (II):

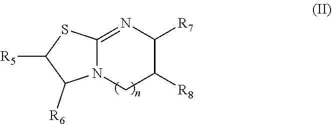

wherein n is 0 to 3;
each of R₅ and R₆ is H, COOR₉, CONHR₉, CONR₉R₉, COR₉, or a substituted or an unsubstituted group selected from alkyl, heteroalkyl, cycloalkyl, heterocycloalkyl, aryl, heteroaryl, alkynyl, alkenyl, heteroarylalkyl, heteroalkylaryl, arylalkenyl, and arylalkyl, wherein R₉ is alkyl or aryl;
each of R₇ and R₈ is H, nitrile, nitro, OR₉, COOR₉, CONHR₉, CONR₉R₉, COR₉, SO₂R₉, or a substituted or an unsubstituted group selected from alkyl, heteroalkyl, cycloalkyl, heterocycloalkyl, aryl, heteroaryl, alkynyl, alkenyl, heteroarylalkyl, heteroalkylaryl, arylalkenyl, and arylalkyl, wherein R₉ is alkyl or aryl; or
R₅ and R₆ and/or R₇ and R₈ are linked together to form an aliphatic, an aromatic, or a heterocyclic ring, each of which is optionally independently substituted with a group selected from alkyl, aryl, heteroaryl, halo, cyano, alkoxy, amino, carboxyl, amide, and ester.

In some embodiments, the glycosyl donor is a glycosyl halide selected from:

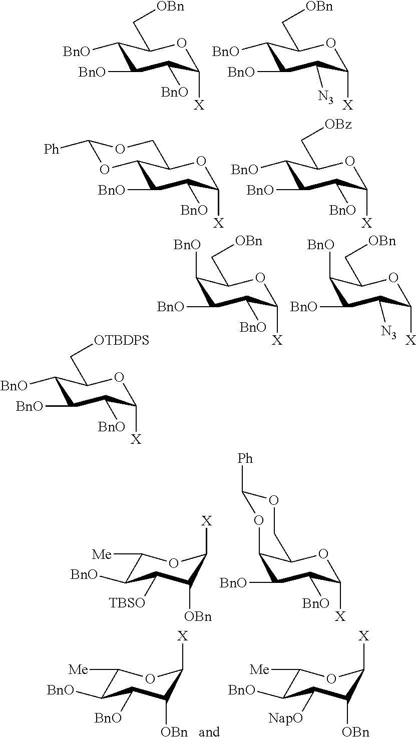

wherein, X is Cl, Br, I, and F.

In some embodiments, the isothiourea catalyst of formula (II) is:

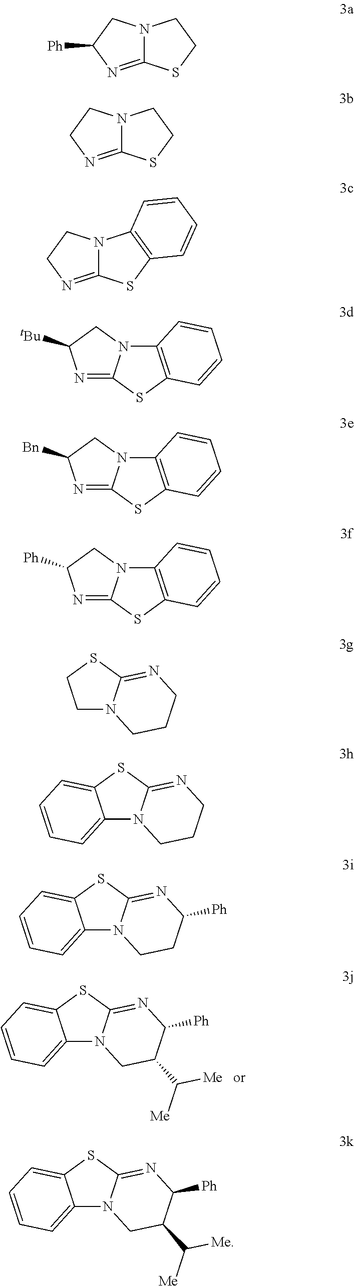

The glycosyl acceptor used for glycosylation can comprise an alcohol, a thiol, or an amine. The glycosyl acceptor can be a monosaccharide, a disaccharide, an oligosaccharide, or a polysaccharide, each comprising at least one —OH, —SH, or a primary or secondary amino group. In some embodiments, the glycosyl acceptor is selected from:

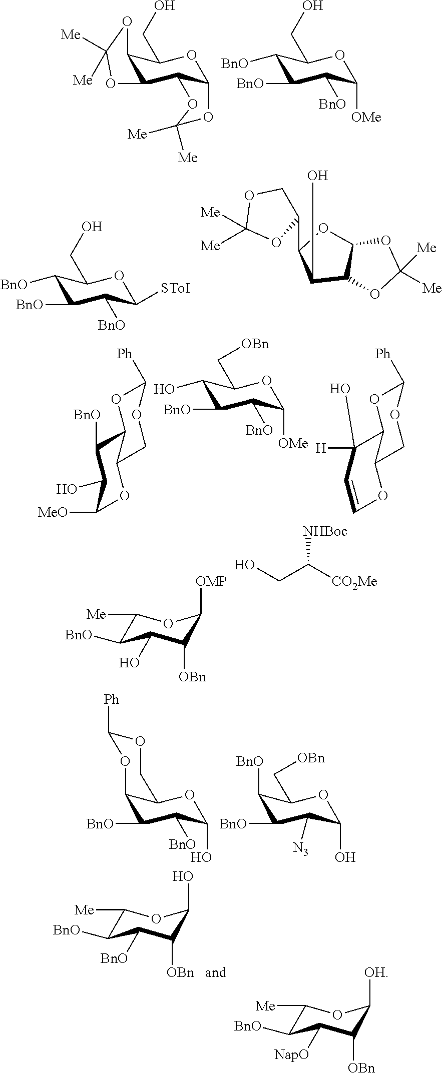

The catalytic stereoselective glycosylation can be carried out in the presence of a base. In some embodiments, the base is selected from triethylamine, tri-tert-butylpyrimidine (TTBP), 2,6-di-tert-butylpyridine, N, N-diisopropyl ethylamine (DIPEA), 1,8-diazabicycloundec-7-ene (DBU), 2, 6-lutidine, and 2,4,6-collidine. Desirably, the base is triethylamine. The catalytic stereoselective glycosylation further comprises the use of an organic solvent. In some embodiments, the organic solvent is selected from dichloromethane, dichloroethane, chloroform, ethyl acetate, toluene, tetrahydrofuran, dimethylformamide, acetone, benzene, and methyl-tert-butyl ether. Desirably, the organic solvent is dichloroethane.

In some embodiments, the α-1,2-cis glycosidic linkage is formed with about 80% to about 99% stereoselectivity. In some embodiments, the α-1,2-cis glycosidic linkage is formed with greater than or equal to 95% stereoselectivity. In some embodiments, the α-1,2-cis glycosidic linkage is formed with greater than or equal to 98% stereoselectivity. In some embodiments, the α-1,2-cis glycosidic linkage is formed with greater than or equal to 99% stereoselectivity.

The catalytic stereoselective glycosylation can be carried out at a temperature from about room temperature to about 100° C. Desirably, the catalytic stereoselective glycosylation is carried out at a temperature of about 65° C.

In some embodiments, the α-1,2-cis glycosidic linkage forms a compound selected from:

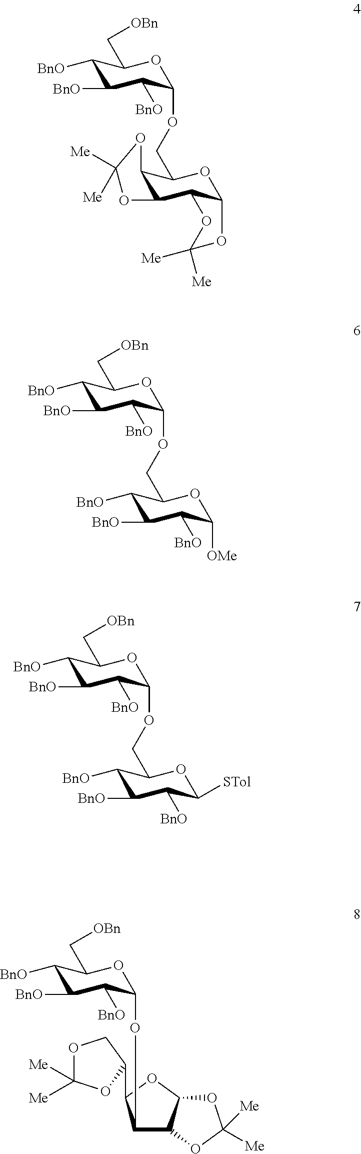

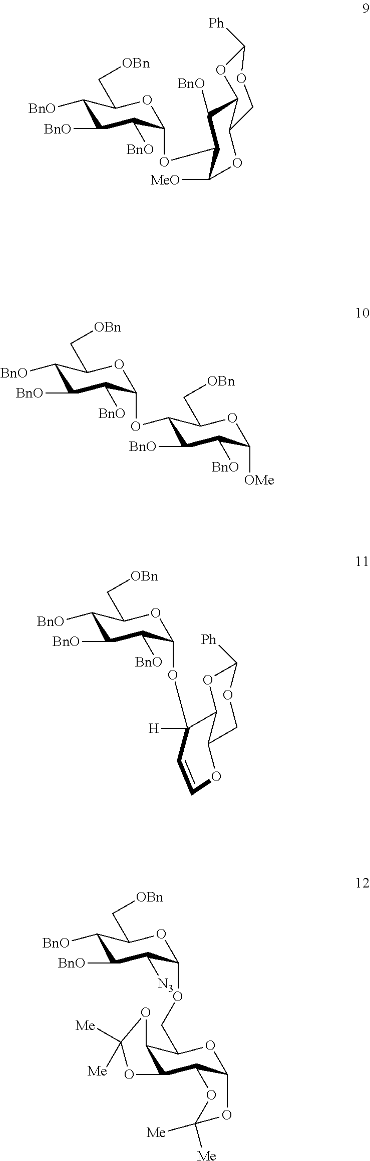

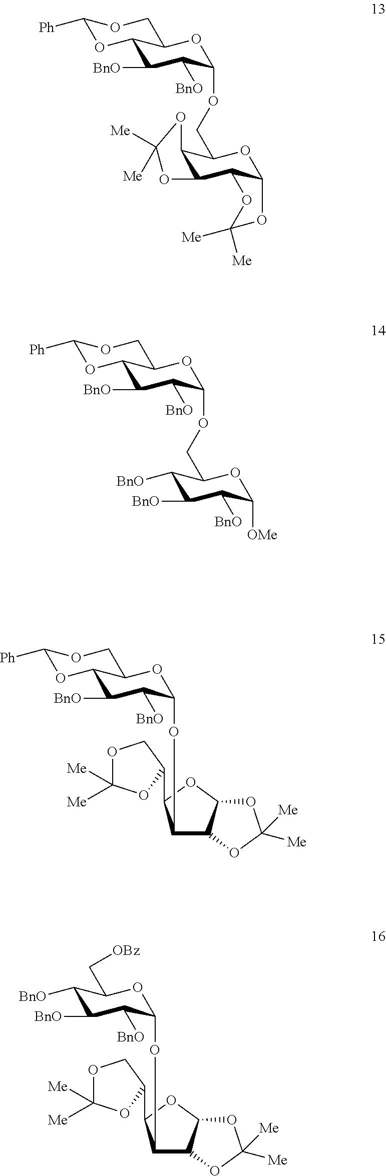

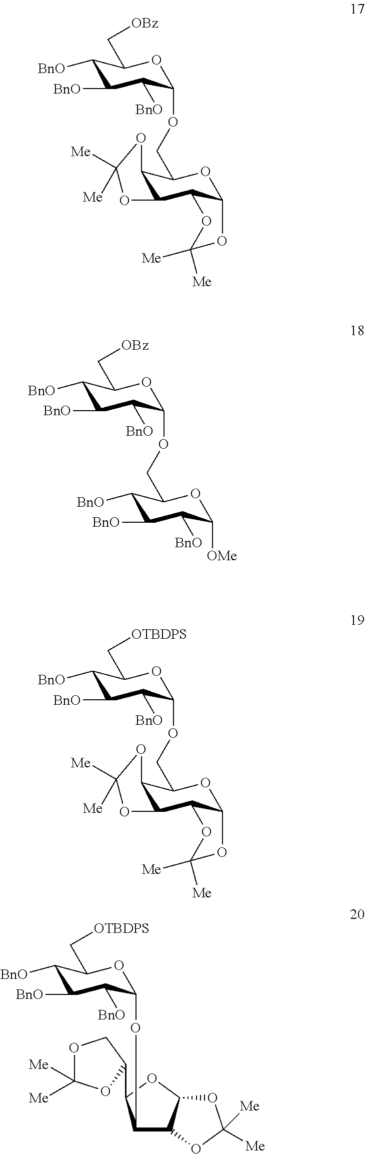

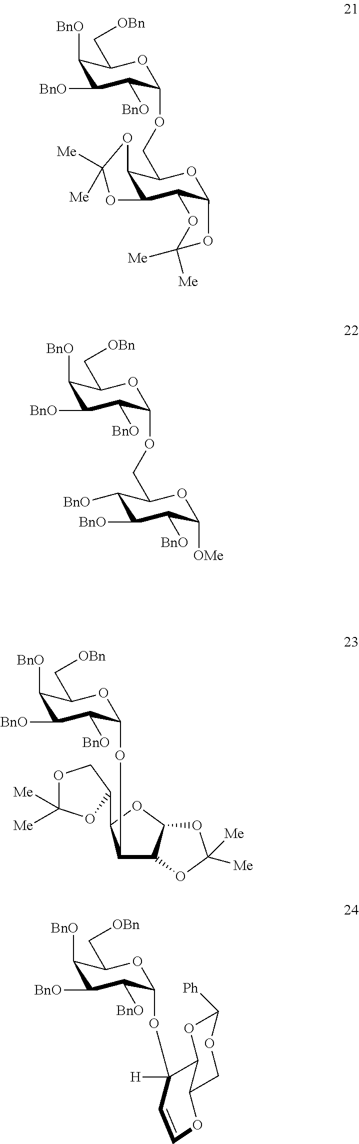

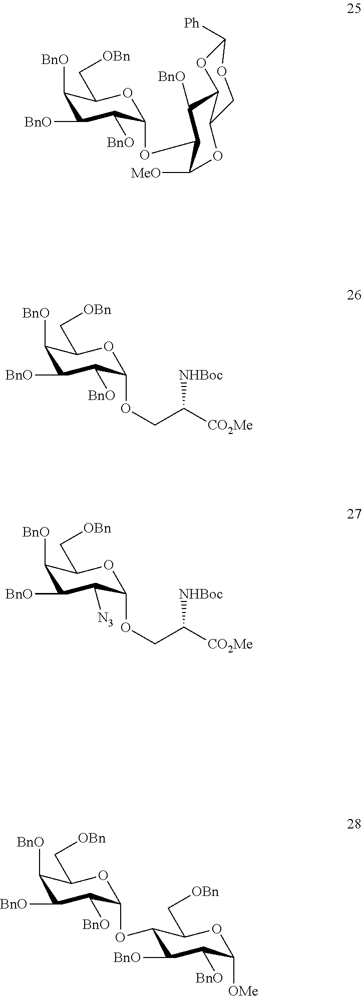

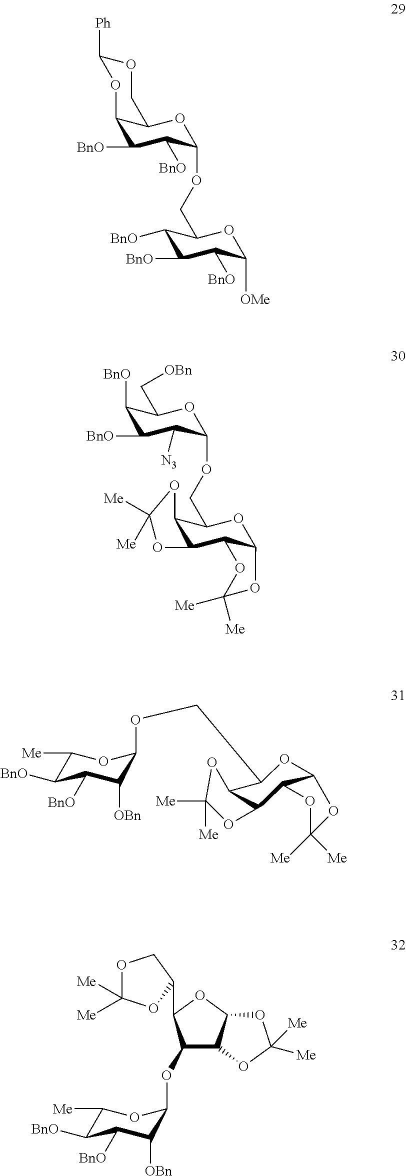

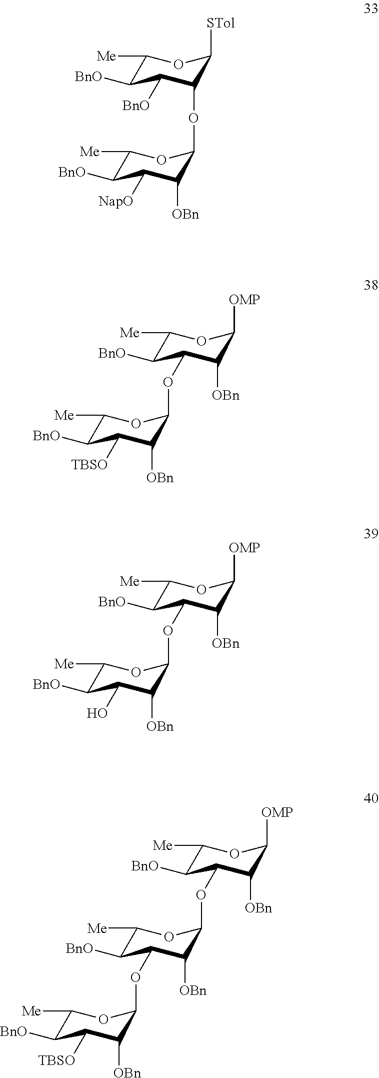

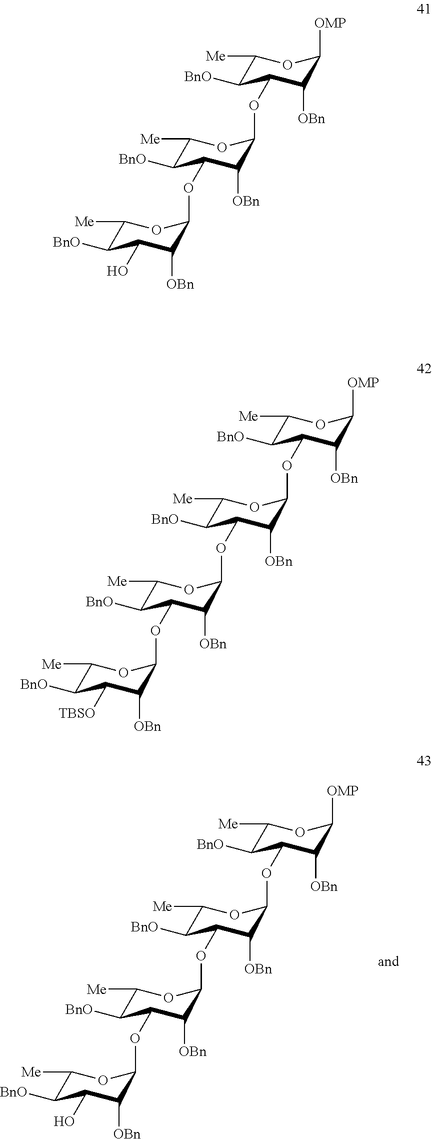

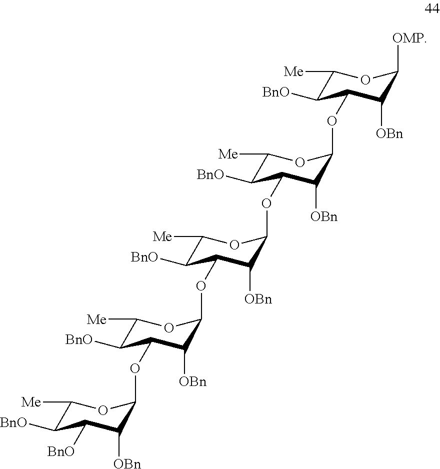

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The term “glycosidic linkage” or “glycosidic bond” refers to a type of ether bond that joins a carbohydrate (sugar) molecule to another group, which may or may not be another carbohydrate.

Abbreviations used are:
Bn—benzyl; Bz—benzoyl; TBDPS—tert-butyldiphenylsilyl; NAP—2-naphthylmethyl; TBAF—tetra-n-butyl ammonium fluoride; Ph—phenyl; Me—methyl; Tol—tolyl; and Boc—tert-butyoxycarbonyl; OMP—paramethoxylphenyl; TBS—tert-butyldimethylsilyl; tert—tertiary.

A method for stereoselective glycosylation of a glycosyl donor, such as glycosyl halide, using an isothiourea catalyst, is provided. The method yields α-1,2-cis saccharides in both excellent yield and stereoselectivity by employing a double S_{N}2 reaction pathway. This method can avoid classical Koenigs-Knorr conditions and thus does not require super stoichiometric use of costly metals, enforces programmable selectivity, and utilizes bench-stable building blocks that can be easily prepared.

Provided is a catalytic stereoselective glycosylation method for preparing an α-1,2-cis glycosidic linkage, which method comprises:
reacting a glycosyl donor of formula (I):

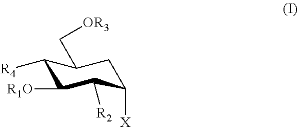

wherein
each of R₁ and R₃ is independently a substituted or an unsubstituted group selected from alkyl, heteroalkyl, cycloalkyl, heterocycloalkyl, aryl, heteroaryl, alkynyl, alkenyl, heteroarylalkyl, heteroalkylaryl, arylalkenyl, allyl, arylalkyl, alkylcarboxyl, arylcarboxyl, trialkylsilyl, diarylalkylsilyl, dialkylarylsilyl, alkoxyalkyl, tetrahydropyranyl, benzyl, and benzoyl, wherein each substituted group can be independently substituted with at least one of alkyl, aryl, heteroaryl, halo, cyano, alkoxy, amino, carboxyl, amide, and ester;
each of R₂ and R₄ is independently alkyl, N₃ or OR₁, wherein R₁ is as defined above; or any two adjacent groups selected from OR₁ and R₂, OR₁ and R₄, and OR₃ and R₄ are linked together to form a 5- to 7-membered cyclic ring, wherein the cyclic ring can be optionally independently substituted with at least one of alkyl, aryl, heteroaryl, halo, cyano, alkoxy, trialkylsilyl, diarylalkylsilyl, amino, carboxyl, amide, and ester group; and
X is a halide selected from Cl, Br, I and F;
with a glycosyl acceptor in the presence of a base and an isothiourea catalyst of formula (II):

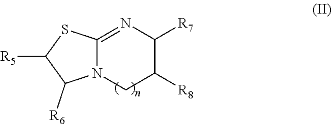

wherein n is 0 to 3;
each of R₅ and R₆ is H, COOR₉, CONHR₉, CONR₉R₉, COR₉, or a substituted or an unsubstituted group selected from alkyl, heteroalkyl, cycloalkyl, heterocycloalkyl, aryl, heteroaryl, alkynyl, alkenyl, heteroarylalkyl, heteroalkylaryl, arylalkenyl, and arylalkyl, wherein R₉ is alkyl or aryl;
each of R₇ and R₈ is H, nitrile, nitro, OR₉, COOR₉, CONHR₉, CONR₉R₉, COR₉, SO₂R₉ or a substituted or an unsubstituted group selected from alkyl, heteroalkyl, cycloalkyl, heterocycloalkyl, aryl, heteroaryl, alkynyl, alkenyl, heteroarylalkyl, heteroalkylaryl, arylalkenyl, and arylalkyl, wherein R₉ is alkyl or aryl; or
R₅ and R₆ and/or R₇ and R₈ are linked together to form an aliphatic, an aromatic, or a heterocyclic ring, each of which is optionally independently substituted with a group selected from alkyl, aryl, heteroaryl, halo, cyano, alkoxy, amino, carboxyl, amide, and ester.

Being canonical electrophiles, glycosyl halides are ideal glycosyl donors in displacement reactions. The anomeric stereochemistry is inverted through a direct S_{N}2 displacement of a leaving group. Thus, in this case of double inversion, first by the catalyst and then by the glycosyl acceptor, the stereochemistry of the glycosyl halide can dictate the stereochemistry of the product.

In some embodiments, the glycosyl donor is a glycosyl halide. The glycosyl halide can be selected from:

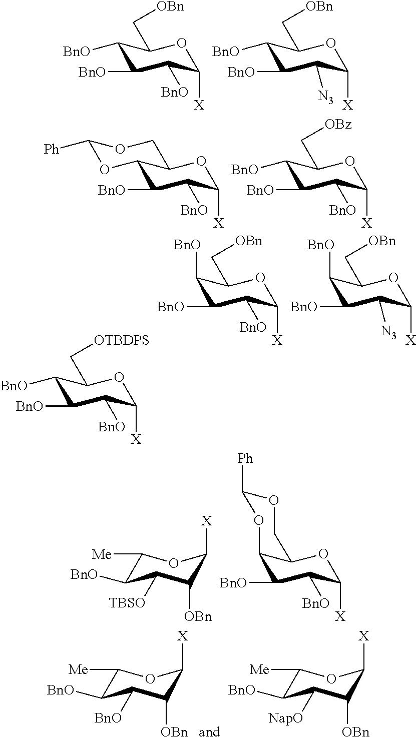

wherein, X is Cl, Br, I, and F.

In some embodiments, the adjacent groups OR₃ and R₄ are linked together to form a 5- to 7-membered cyclic ring, wherein the cyclic ring can be optionally independently substituted with at least one of alkyl, aryl, heteroaryl, halo, cyano, alkoxy, trialkylsilyl, diarylalkylsilyl, amino, carboxyl, amide, and ester group.

Provided is a catalyst for a stereoselective method of glycosylation. Isothioureas are known to be highly nucleophilic acyl transfer catalysts. Isothiourea engagement with the glycosyl halide can form an equatorially disposed cationic intermediate, which can be intercepted by a glycosyl acceptor forming a new alpha-linked disaccharide and releasing the catalyst. This double S_{N}2 strategy can relay the stereochemistry of the starting glycosyl halide into the product (see Scheme 1).

In some embodiments, the isothiourea catalyst is:

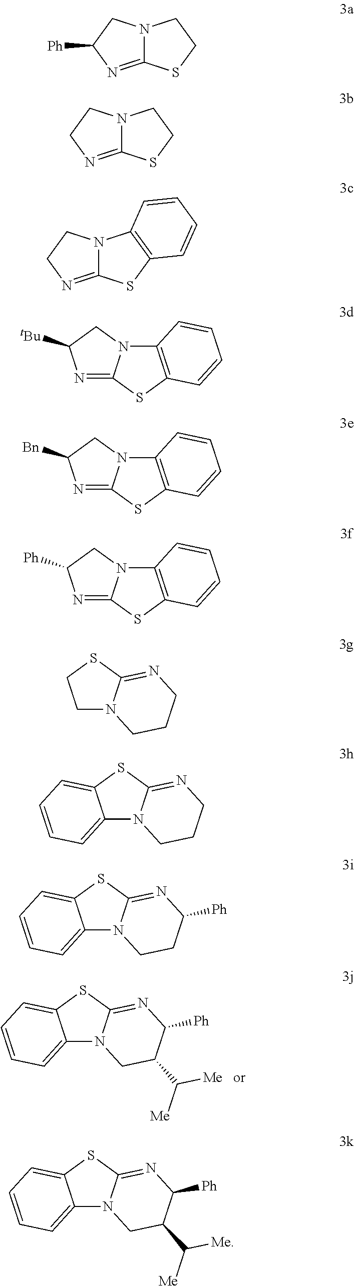

Any suitable glycosyl acceptor, which comprises an alcohol, a thiol, or an amine group, can be used for the stereoselective glycosylation of a glycosyl halide. The glycosyl acceptor can be a monosaccharide, a disaccharide, an oligosaccharide, or a polysaccharide, each comprising at least one of —OH, —SH, or a primary or secondary amino group.

In some embodiments, the glycosyl acceptor is selected from:

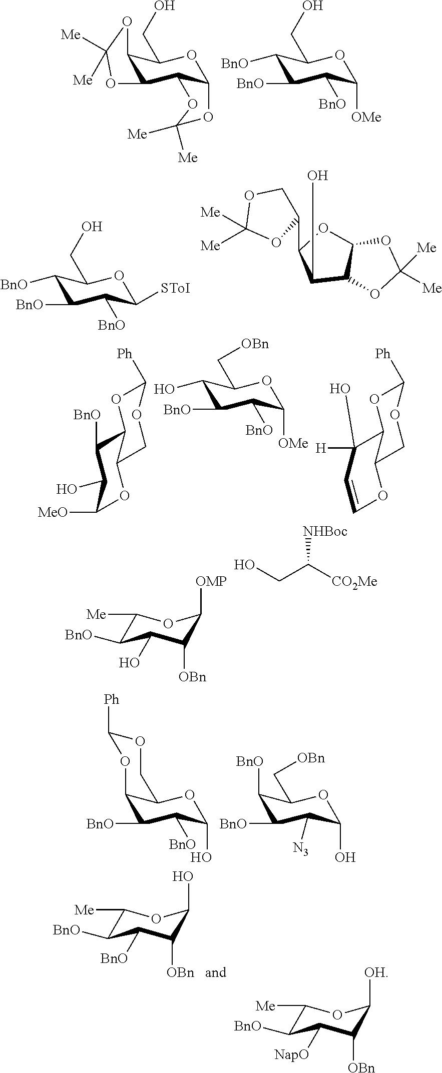

Any suitable base can be used for the catalytic stereoselective glycosylation method. Examples of the bases include, but are not limited to, triethylamine, tri-tert-butylpyrimidine (TTBP), 2,6-di-tert-butylpyridine, N,N-diisopropyl ethylamine (DIPEA), 1,8-diazabicycloundec-7-ene (DBU), 2,6-lutidine, and 2,4,6-collidine. In some embodiments, the base is triethylamine.

The method can be carried out using an organic solvent. Any suitable organic solvent can be used. Examples of suitable organic solvents include, but are not limited to, dichloromethane, dichloroethane, chloroform, ethyl acetate, dimethyl formamide, toluene, acetone, benzene, methyl tert-butyl ether, and tetrahydrofuran. A desirable organic solvent is dichloroethane.

In some embodiments, the catalytic stereoselective glycosylation can be carried out at a temperature from about room temperature (RT) to about 100° C., such as from about RT to 100° C., RT to about 100° C., or RT to 100° C. Desirably, the catalytic stereoselective glycosylation can be carried out at about 65° C. (such as 65° C.).

The catalytic stereoselective glycosylation method comprises the formation of a glycosidic bond with about 80% to about 99% stereoselectivity for an α-linkage. The method can yield the alpha anomer as the predominant or sole product. In some embodiments, the α-1,2-cis glycosidic linkage is formed with greater than or equal to 95% stereoselectivity. In some embodiments, the α-1,2-cis glycosidic linkage is formed with greater than or equal to 98% stereoselectivity. In some embodiments, α-1,2-cis glycosidic linkage is formed with greater than or equal to 99% stereoselectivity.

In some embodiment, the α-1,2-cis glycosidic linkage forms a compound selected from:

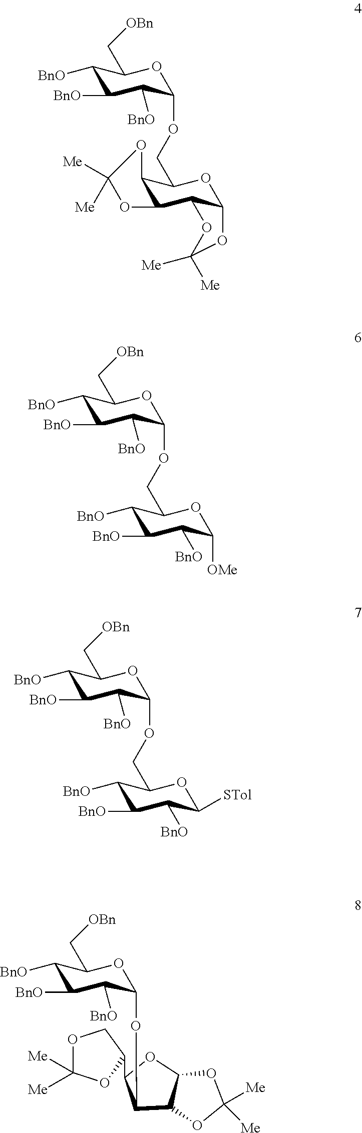

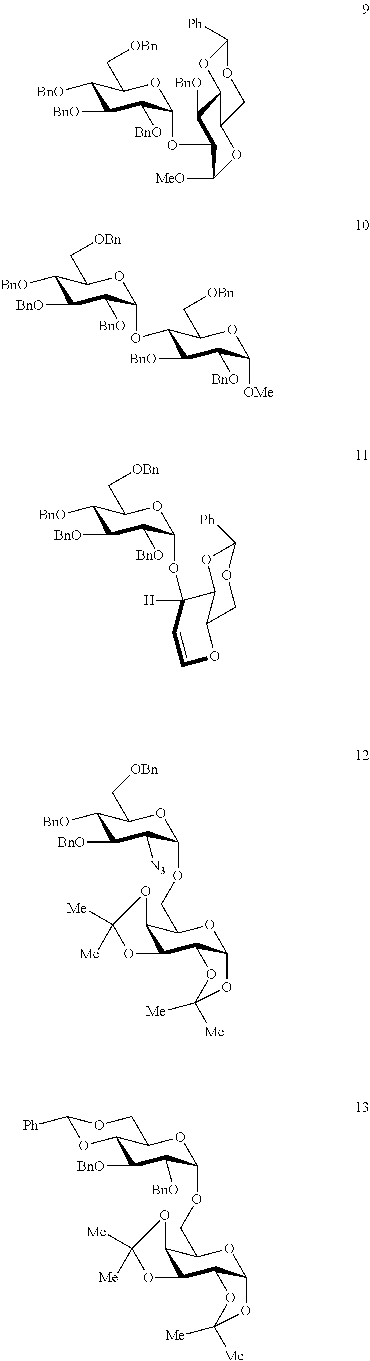

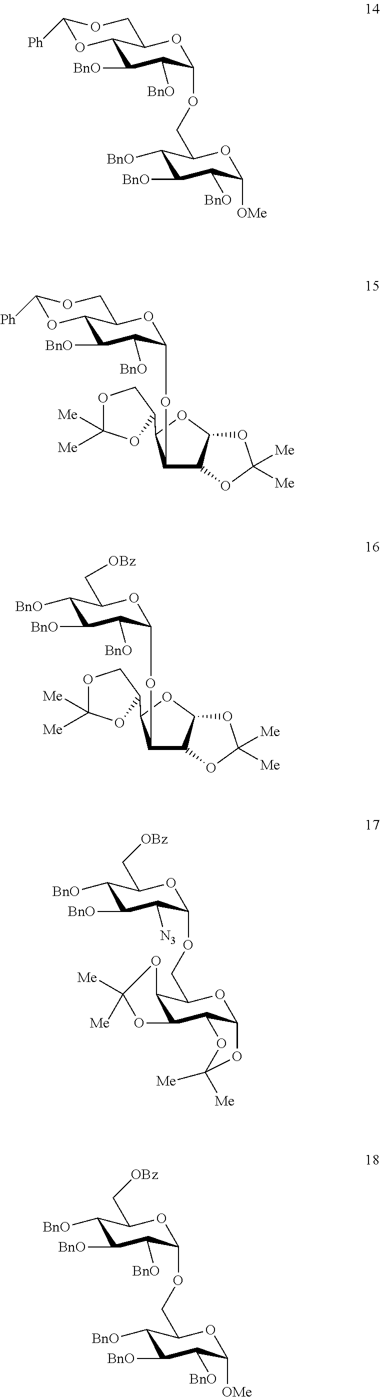

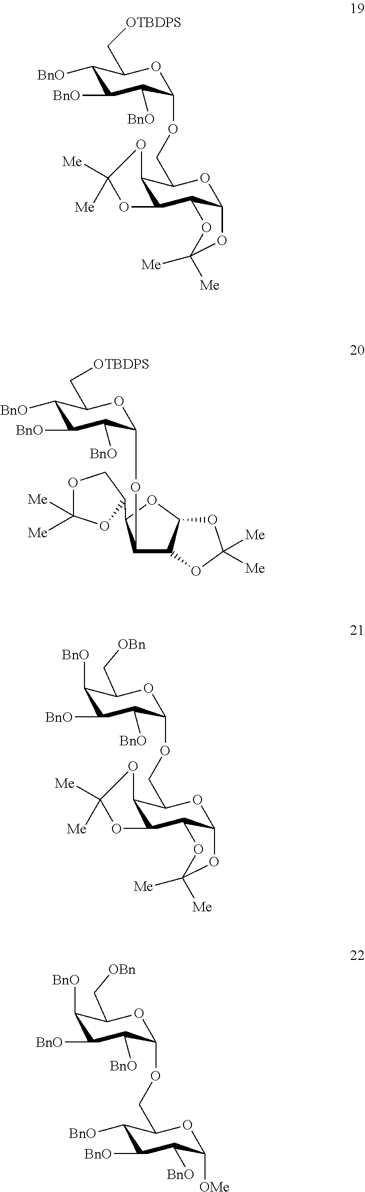

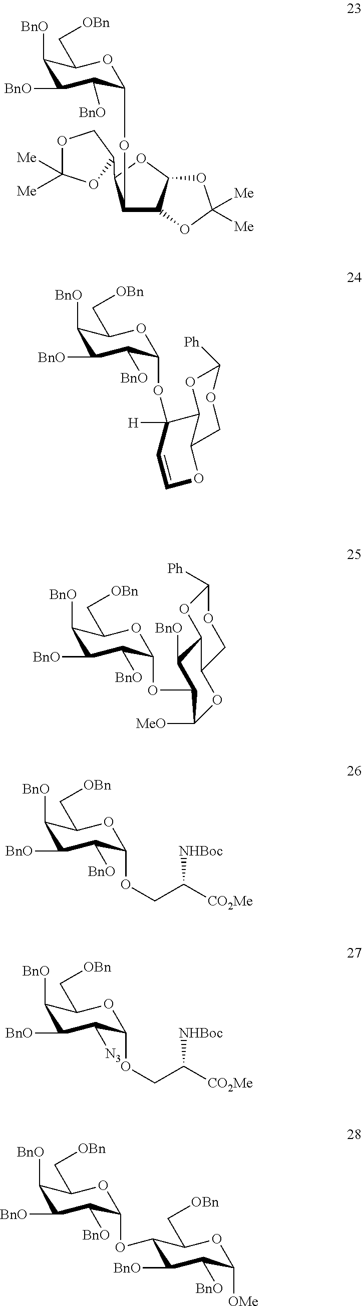

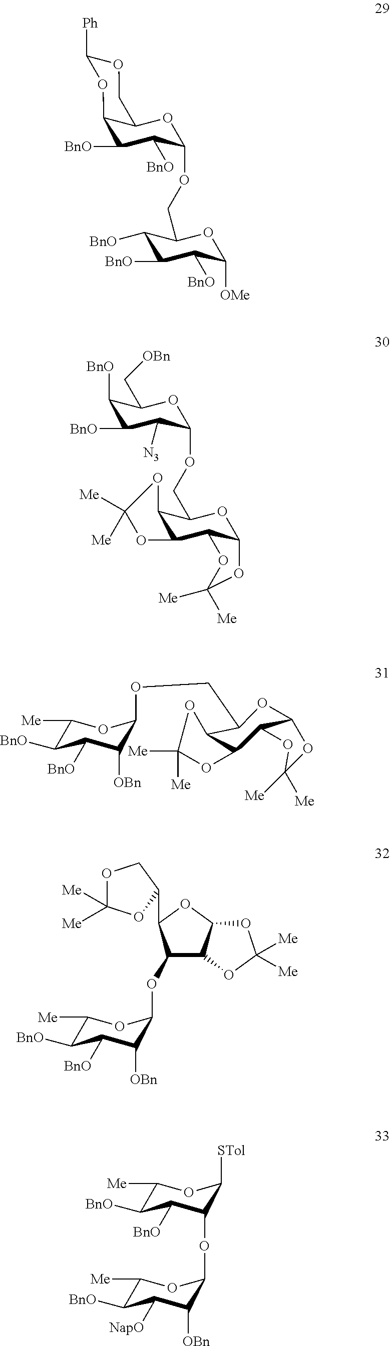

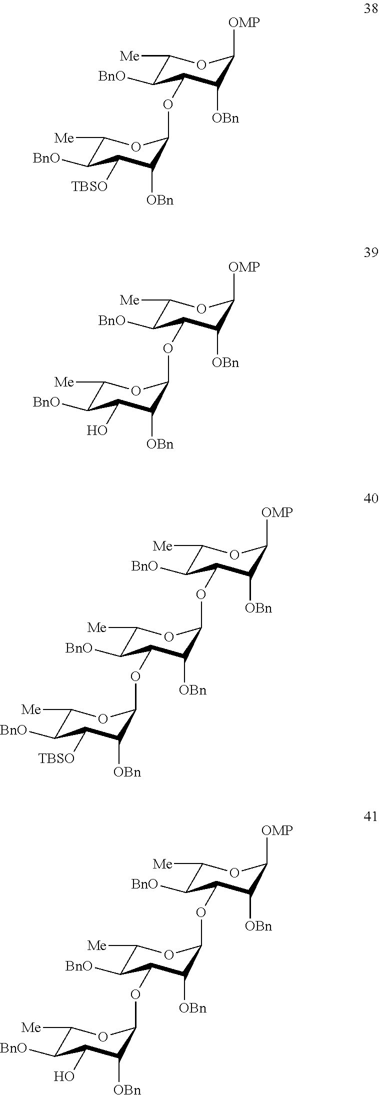

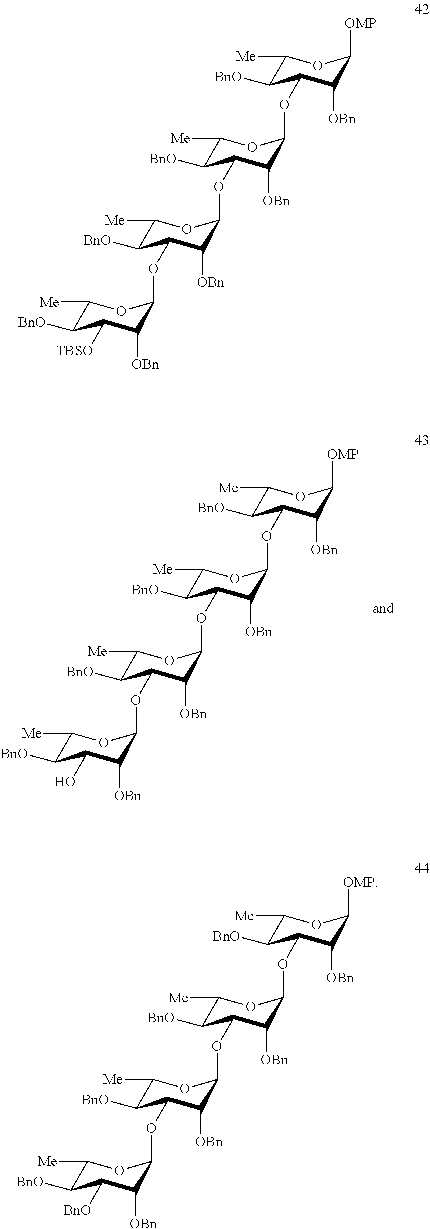

TABLE 1
summarises the yield and stereoselectivity data of the glycosylation of a
glycosyl halide in the presence of isothiourea catalysts selected from 3a-3k.

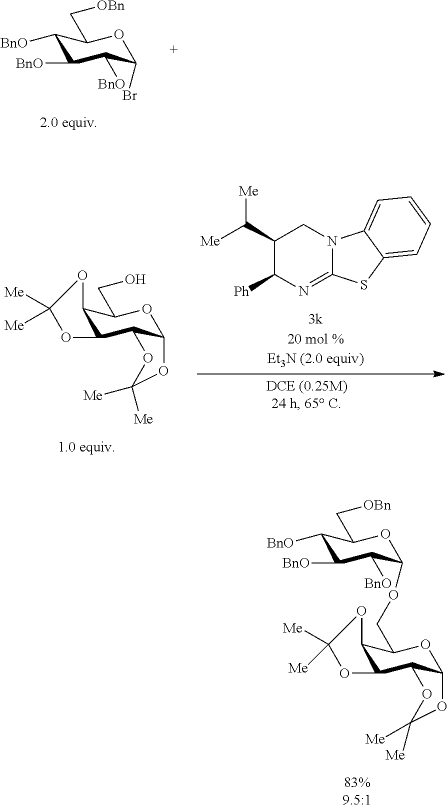

| Entry | Deviation from Standard Conditions | Yield, a/b |
|---|---|---|
| 1 | none | 83%, 9.5:1 |
| 2 | catalyst 3a | 48%, 5:1 |
| 3 | catalyst 3b | 57%, 4.5:1 |
| 4 | catalyst 3c | 67%, 5.2:1 |
| 5 | catalyst 3d | 75%, 5.2:1 |
| 6 | catalyst 3e | 78%, 4.3:1 |
| 7 | catalyst 3f | 61%, 6:1 |
| 8 | catalyst 3g | 55%, 6:1 |
| 9 | catalyst 3h | 71%, 6:1 |
| 10 | catalyst 3i | 57%, 4.5:1 |
| 11 | catalyst 3j | 74%, 4.5:1 |

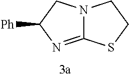 [SMILES: c1ccc([C@H]2CN3CCSC3=N2)cc1]

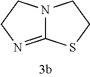

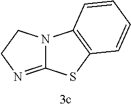 [SMILES: c1ccc2c(c1)SC1=NCCN12]

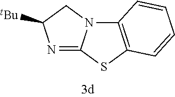 [SMILES: CC(C)(C)[C@H]1CN2C(=N1)Sc1ccccc12]

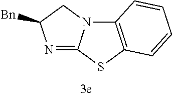 [SMILES: c1ccc(C[C@H]2CN3C(=N2)Sc2ccccc23)cc1]

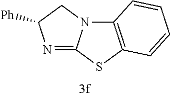 [SMILES: c1ccc([C@@H]2CN3C(=N2)Sc2ccccc23)cc1]

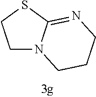

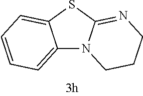 [SMILES: c1ccc2c(c1)SC1=NCCCN12]

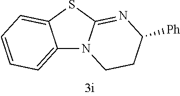 [SMILES: c1ccc([C@@H]2CCN3C(=N2)Sc2ccccc23)cc1]

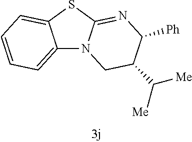 [SMILES: CC(C)[C@@H]1CN2C(=N[C@@H]1c1ccccc1)Sc1ccccc12]

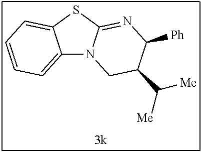 [SMILES: CC(C)[C@H]1CN2C(=N[C@H]1c1ccccc1)Sc1ccccc12]

The glycosidic bond formation using the catalyst isothiourea was studied with various glycosyl donors. Catalyst 3k and glucosyl bromide 1 were reacted stoichiometrically to form adduct 5. This was characterized and confirmed through HRMS, NMR, and ESI with an m/z ratio of 831.3667. The adduct 5 was subjected to glycosylation with the glycosyl acceptor 2 in the presence of a base, which smoothly reacted to form disaccharide 4 with no erosion of yield or selectivity to form the cis 1,2-alpha glycoside (see Scheme 1).

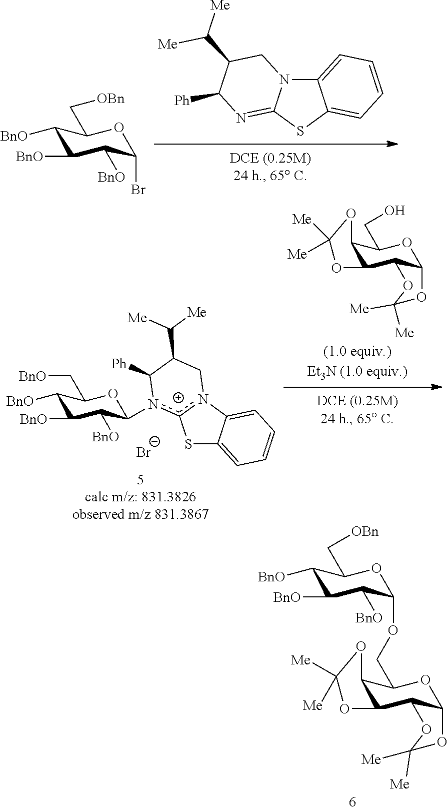

A substrate scope with respect to both the glycosyl donor and the acceptor structures was studied. The C-2 hydroxyl position is the most powerful locus capable of modulating alpha/beta selectivity through ester participation and in all substrates, subsequently, benzyl was protected. Further, we specifically chose benzyl, benzylidene, silyl, and benzoate groups at other hydroxyls to mimic the standard sets of protecting groups commonly found in glycan synthesis. Exposure of 1 to primary acceptors furnished disaccharides 6-8 in good yields, and with good alpha selectivity. With secondary acceptors, including challenging C-2 and C-4 hydroxyls, excellent selectivity was observed in the formation of compound 9 and compound 10. Further, both glycals and 2-deoxy-2-azido sugars were tolerated, forming disaccharides 11 and 12 in good yield, albeit at more modest levels of selection.

Due to the flexibility of monosaccharides and the impact of conformational interconversion on selectivity, switching protecting group strategies can confer a greater degree of alpha selectivity through limiting torsional plasticity. Using this strategy with the C-4 and C-6 hydroxyls protected as a benzylidene. Augmented levels of stereoselection with both primary and secondary acceptors, furnishing disaccharides 13-15 in good chemical yields and in good to complete alpha selectivity were observed. These results were in line with previous observations that conformationally restricted donors operate in a more S_{N}2 like fashion and less driven by oxocarbenium character. Glycosylation with C-6 O-benzoyl and C-6 O-silyl protected donors was investigated, to determine how electron-withdrawing or sterically bulky donors affect glycosylation. Both primary and secondary acceptors were found to be competent nucleophiles, reacting with the benzoylated donor to furnish compound 16-18 in good yields and selectivity. The presence of the C-6 O-TBDPS group attenuated reactivity, as reflected in slightly lower chemical yields of compound 19 and compound 20, but levels of selectivity were comparable to previous donors.

Scheme 1 illustrates the double S_{N}2 reaction, which transmits the glycosyl donor stereochemistry into the product.

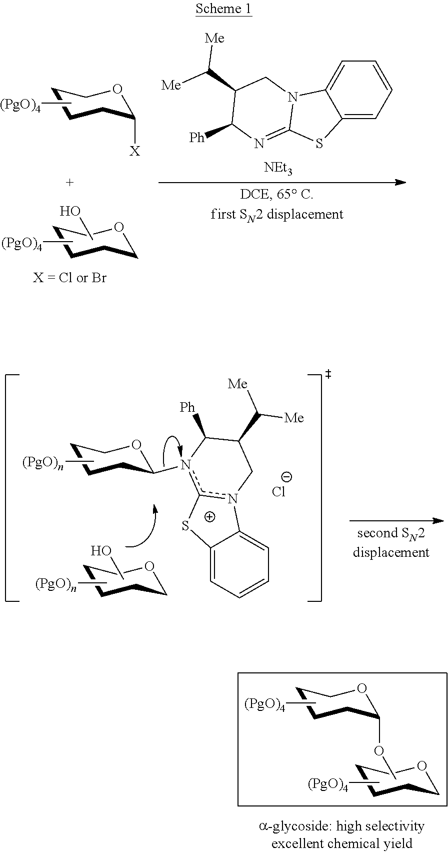

The glycosyl donor of formula (I) can be a glucose donor, a galactose donor, a rhamnose donor, a xylose donor, a arabinose donor, or a fucose donor. The reactivity of galactosyl donors were examined, which, in contrast to the glucose series, reacted as their glycosyl chlorides. Perbenzylated galactosyl chlorides smoothly reacted with both primary and secondary acceptors using the standardized method conditions, forming disaccharides 21-24 in good yields and with complete alpha selectivity. This is remarkable, considering reports of C-4 benzylated D-galactose inverts the expected sense of selectivity. Further, more sterically encumbered acceptors were examined, which were both well tolerated and yielded compounds 25 and 28 in excellent selectivities. Benzylidene protection did not alter selectivity, as evidenced in compound 29. Protected amino acids were found to react smoothly to furnish alpha glycoside 26 and mucin antigen-like structure could be realized in high fidelity.

Following the investigation of glucose and galactose donors, rhamnosylation was studied, which proceeded through the use of the chloride donor. Gratifyingly, primary and secondary acceptors were tolerated well to provide compounds 31 and 32 both in complete alpha selectivity. Further, rhamnose-rhamnose glycosylations were examined, furnishing disaccharide 33 with complete alpha selectivity and in very good yield.

The polysaccharide, such as a disaccharide, trisaccharide, tetrasaccharide, and a pentasaccharide, can be prepared through glycosylation of a glycosyl donor with glycosyl acceptor, for example, disaccharide 38 can be prepared in 76% yield as a single alpha anomer. Removal of the silyl protecting group through treatment with tetra-n-butyl ammonium fluoride (TBAF) provided acceptor 39 in 91% yield. Exposure of compound 39 to the acceptor provided trisaccharide 40 in 78% yield as a single anomer. Following silyl deprotection, and isolation of trisaccharide acceptor 41, glycosylation with generated tetrasaccharide 42 as a single anomer in 70% yield over two steps. Deprotection, followed by glycosylation furnished pentasaccharide 44 as a single anomer in 85% yield over two steps.

The term “substituted” (e.g., as in “optionally substituted”) refers to a functional group in which one or more hydrogen atoms contained therein are replaced by one or more non-hydrogen atoms. The term “functional group” or “substituent” refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, arylalkyloxy groups, oxo(carbonyl) groups, and carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” refers to substituted or unsubstituted straight chain and branched alkyl groups and cycloalkyl groups having from 1 to about 20 carbon atoms (e.g., C₁-C₂₀), 1 to 12 carbons (e.g., C₁-C₁₂), 1 to 8 carbon atoms (e.g., C₁-C₈), or, in some embodiments, from 1 to 6 carbon atoms (e.g., C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. The term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” refers to substituted or unsubstituted straight chain and branched divalent alkenyl and cycloalkenyl groups having from 2 to 20 carbon atoms (e.g., C₂-C₂M), 2 to 12 carbons (e.g., C₂-C₁₂), 2 to 8 carbon atoms (e.g., C₂-C₈) or, in some embodiments, from 2 to 4 carbon atoms (e.g., C₂-C₄) and at least one carbon-carbon double bond. Examples of straight chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

The term “alkynyl” refers to an unsaturated monovalent chain of carbon atoms, including at least one triple bond, which may be optionally branched. In various embodiments that include alkynyl, illustrative examples include lower alkynyl, such as C₂-C₆, C₂-C₄ alkynyl, and the like.

The term “hydroxyalkyl” refers to alkyl groups as defined herein and substituted with at least one hydroxyl (—OH) group.

The term “cycloalkyl” refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (e.g., C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl, cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and cryloyl groups and the like. When the group containing the carbon atom that is bonded to the carbonyl carbon atom contains a halogen, the group is termed a “haloacyl” group. An example is a trifluoroacetyl group.

The term “aryl” refers to substituted or unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (e.g., C₆-C₁₄) or from 6 to 10 carbon atoms (e.g., C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The terms “aralkyl” and “arylalkyl” refer to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” refers to substituted or unsubstituted aromatic and non-aromatic ring compounds containing three or more ring members, of which one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups can include 3 to 8 carbon atoms (e.g., C₃-C₈), 3 to 6 carbon atoms (e.g., C₃-C₆) or 6 to 8 carbon atoms (e.g., C₆-C₈).

A heteroaryl ring is an embodiment of a heterocyclyl group. The phrase “heterocyclyl group” includes fused ring species including those that include fused aromatic and non-aromatic groups. Representative heterocyclyl groups include, but are not limited to, pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-ylpropyl.

The term “heteroarylalkyl” refers to alkyl groups as defined herein in which a hydrogen or carbon bond of an alkyl group is replaced with a bond to a heteroaryl group as defined herein.

The term “alkoxy” refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The terms “halo,” “halogen,” and “halide” group, by themselves or as part of another substituent, mean, unless otherwise stated, a fluorine, chlorine, bromine, or iodine atom. The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

The terms “optionally substituted” and “optional substituents” are used to describe groups, which are either unsubstituted or substituted with one or more of the substituents specified. When the groups in question are substituted with more than one substituent, the substituents can be the same or different. The terms “independently” “independently are” and “independently selected from” mean that the groups in question may be the same or different. Certain of the defined groups or substituents can occur more than once in the structure, and upon such occurrence, each group or substituent shall be defined independently of the other.

It will be appreciated by persons skilled in the art that the present disclosure is not limited by what has been particularly shown and described herein above. Rather the scope of the present disclosure includes both combinations and sub-combinations of the various features described herein above as well as variations and modifications which would occur to persons skilled in the art upon reading the specification and which are not in the prior art.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

### EXAMPLES

### General Information

All reactions were performed in flame dried round bottom or modified Schlenk (Kjedahl shape) flasks fitted with rubber septa or a yellow PTFE cap under a positive pressure of argon, unless otherwise indicated. Air- and moisture-sensitive liquids and solutions were transferred by syringe or canula. Dry THF, Et₂O and CH₂Cl₂ were obtained from a PureSolv MD-5 Solvent Purification System (Inert). All other reagents were used as obtained from commercial sources without further purification. Analytical thin-layer chromatography (TLC) was carried out using 0.2 mm commercial silica gel plates (silica gel 60, F254, Merck) and visualized using a UV lamp and or ceric ammonium molybdate (CAM) or aqueous potassium permanganate (KMnO₄) stain. Preparatory TLC was also carried out on the same silica gel plates. Organic solutions were concentrated using a Heidolph rotary evaporator at ˜10 torr.

NMR spectra were recorded on Bruker spectrometers (¹¹H at 500 MHz and ¹³C at 125 MHz). Chemical shifts (δ) are given in ppm with reference to residual proton signals in the solvent [¹H NMR-CHCl₃ (7.26); ¹³C NMR: CDCl3 (77.00). Data are presented as follows: chemical shift, multiplicity (s=singlet, d=doublet, t=triplet, q=quartet, m=multiplet, and bs=broad singlet), integration, and coupling constant in hertz (Hz). High-resolution mass (HRMS) measurements for compound characterization were carried out using a Quan TOF analyzer or an Agilent 6550 QTOF system and are reported as m/z (relative intensity). Infrared spectra were recorded on ThermoFisher Nicolet iS50 FT IR using neat thin film technique.

### Examples

### General Procedure A

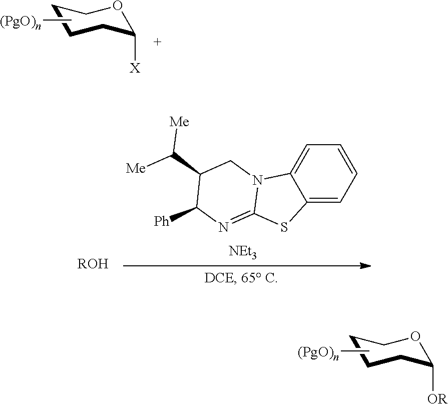

A 10 mL flame-dried round bottle flask was charged with glucosyl bromide 1a (0.4 mmol, 2.0 equiv.), alcohol (0.2 mmol, 1.0 equiv.), catalyst 3k (0.04 mmol, 20 mol %), Et₃N (0.4 mmol, 2.0 equiv.) and DCE (0.8 mL). The reaction was stirred at 65° C. for 20-24 h, concentrated, and purified by silica gel flash chromatography (hexanes/ethyl acetate) to give the desired product.

### General Procedure B

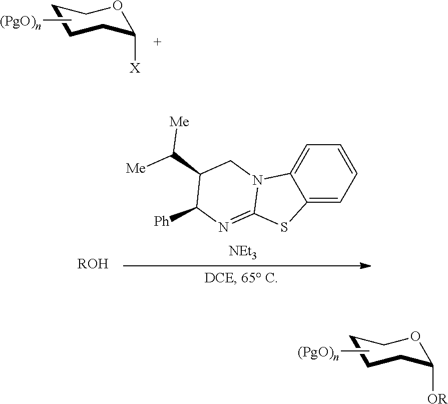

A 10 mL flame-dried round bottle flask was charged with galactosyl or rhamnosyl chloride (0.4 mmol, 2.0 equiv), alcohol (0.2 mmol, 1.0 equiv), catalyst 3k (0.04 mmol, 20 mol %), Et₃N (0.4 mmol, 2.0 equiv.) and DCE (0.4 mL). The resulting solution was stirred at 65° C. for 18-24 h, concentrated, and purified by silica gel flash chromatography (hexanes/ethyl acetate) to give the desired product.

The α/β ratio of the desired products were determined by ¹H NMR analysis based on the ratio of the anomeric protons of both α- and β-anomers. When the anomeric protons are overlapped, other diagnostic protons of both anomers were analyzed.

### General Procedure C

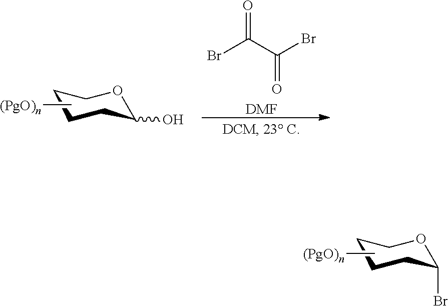

To a stirred (0.1M DCM) solution of hemiacetal at 23° C. was added DMF (1.5 equiv.). To this was added a solution of oxalyl bromide (2.0M DCM, 4.0 equiv.), dropwise. Upon consumption of starting material, the reaction was poured into saturated aqueous NaHCO₃, and extracted three times with 1:1 hexanes:ethyl acetate. The combined organic layers were washed with brine, dried over sodium sulfate and concentrated, in vacuo. The product was used immediately and was sufficiently pure.

### General Procedure D

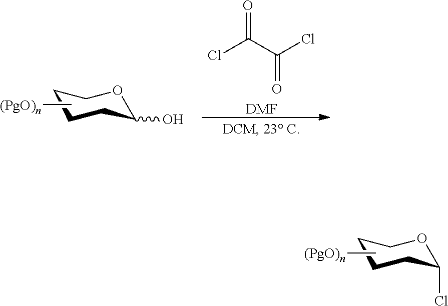

To a stirred (0.1M DCM) solution of hemiacetal at 23° C. was added DMF (1.5 equiv.). To this was added neat oxalyl chloride (4.0 equiv.), dropwise. Upon consumption of starting material, the reaction was poured into saturated aqueous NaHCO₃, and extracted three times with 1:1 hexanes:ethyl acetate. The combined organic layers were washed with brine, dried over sodium sulfate and concentrated, in vacuo. Silica gel chromatography (hexanes:ethyl acetate) furnished the glycosyl chlorides.

### Experimental Data

### Example 1: Compound 4

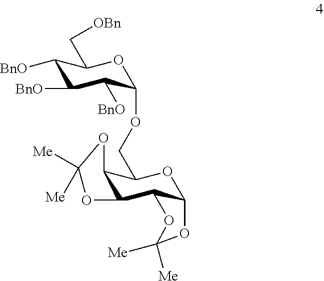

α anomer of 4 was prepared using general Procedure A. Yield: 130 mg, 83%; α:β=9.5:1; R_{f}=0.39 (hexane/AcOEt 4:1);

¹H NMR (500 MHz, CDCl₃): δ 7.40-7.23 (m, 18H), 7.17-7.12 (m, 2H), 5.53 (d, J=5.0 Hz, 1H), 5.01 (d, J=3.7 Hz, 1H), 4.99 (d, J=10.9 Hz, 1H), 4.82 (dd, J=12.2, 10.8 Hz, 2H), 4.75 (d, J=11.9 Hz, 1H), 4.70 (d, J=11.9 Hz, 1H), 4.63 (d, J=12.1 Hz, 1H), 4.60 (dd, J=7.9, 2.4 Hz, 1H), 4.48 (dd, J=11.5, 8.4 Hz, 2H), 4.36 (dd, J=7.9, 1.9 Hz, 1H), 4.32 (dd, J=5.0, 2.4 Hz, 1H), 4.05 (ddd, J=7.9, 6.1, 1.9 Hz, 1H), 3.99 (t, J=9.3 Hz, 1H), 3.83 (dt, J=9.9, 2.7 Hz, 1H), 3.81-3.73 (m, 3H), 3.68 (dd, J=10.1, 9.0 Hz, 1H), 3.65 (dd, J=10.7, 2.1 Hz, 1H), 3.59 (dd, J=9.6, 3.6 Hz, 1H), 1.53 (s, 3H), 1.45 (s, 3H), 1.33 (s, 3H), 1.31 (s, 3H);

¹³C NMR (126 MHz, CDCl₃): δ 138.90, 138.31, 137.98, 128.61, 128.32, 127.90, 127.85, 127.79, 127.66, 127.61, 127.48, 109.17, 108.57, 97.01, 96.27, 81.93, 79.76, 77.53, 75.60, 74.96, 73.43, 72.31, 70.77, 70.61, 70.18, 68.31, 66.16, 65.65, 29.67, 26.14, 26.04, 24.89, 24.61;

IR (cm⁻¹): 2922, 1724, 1496, 1453, 1371, 1255, 1209, 1163, 1066, 1027, 999, 918, 889, 735, 696, 511;

HRMS-ESI (m/z): [M+H]⁺ calcd. For [C₄₆H₅₄O₁₁]⁺ 783.3666. found 805.3556, [M+Na]⁺.

### Example 2: Compound 6

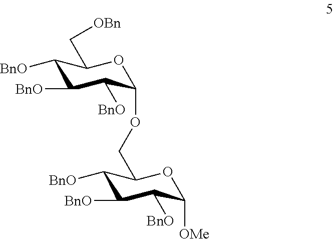

α anomer of δ was prepared using general Procedure A. Yield: 144 mg, 73%; α:β=8:1; R_{f}=0.45 (hexane/AcOEt=4:1);

¹H NMR (500 MHz, CDCl₃): δ 7.44-7.16 (m, 35H), 5.08-4.97 (m, 4H), 4.93-4.83 (m, 3H), 4.81-4.70 (m, 4H), 4.67-4.61 (m, 3H), 4.56-4.46 (m, 2H), 4.10-4.01 (m, 2H), 3.93-3.83 (m, 3H), 3.82-3.66 (m, 4H), 3.65-3.58 (m, 2H), 3.52 (dd, J=9.6, 3.6 Hz, 1H), 3.43 (s, 3H);

¹³C NMR (126 MHz, CDCl₃): δ 138.78, 138.42, 138.39, 138.14, 137.94, 128.37, 128.32, 128.29, 127.97, 127.84, 127.69, 127.58, 97.92, 97.21, 82.10, 81.64, 80.10, 79.94, 77.73, 77.57, 75.68, 75.46, 74.93, 73.34, 72.31, 70.32, 70.19, 78.42, 66.00, 66.1, 55.11;

IR (cm⁻¹): 3030, 2920, 1496, 1452, 1366, 1153, 1071, 1039, 1026, 996, 937, 735, 694, 611, 466;

HRMS-ESI (m/z): [M+Na]⁺ calcd. For [C₆₂H₆₆NaO₁₁]⁺ 1009.4497. found 1009.4494.

### Example 3: Compound 7

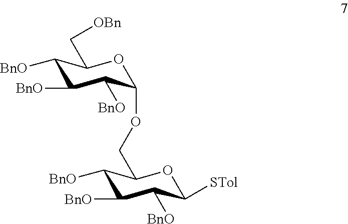

α anomer of 7 was prepared using general Procedure A. Yield: 139 mg, 73%; α:β=20:1; R_{f}=0.45 (hexane/AcOEt=5:1);

¹H NMR (500 MHz, CDCl₃): δ 7.46 (d, J=10.0 Hz, 2H), 7.41-7.23 (m, 33H), 7.14-7.12 (m, 2H), 7.07 (d, J=10.0 Hz, 2H), 5.04 (d, J=5.0 Hz, 1H), 5.00-4.52 (m, 14H), 3.99 (t, J=10.0 Hz, 1H), 3.89-3.83 (m, 2H), 3.78-3.58 (m, 8H), 3.49-3.46 (m, 1H), 3.25 (t, J=10.0 Hz, 1H), 2.23 (s, 3H);

¹³C NMR (126 MHz, CDCl₃): δ 138.87, 138.54, 138.48, 138.44, 138.18, 138.08, 138.04, 137.99, 137.75, 132.88, 129.76, 128.43, 128.38, 128.34, 128.22, 127.95, 127.88, 127.80, 127.72, 127.65, 127.62, 127.51, 97.34, 88.45, 86.64, 81.75, 81.09, 80.13, 78.72, 77.64, 75.63, 75.43, 74.94, 73.38, 72.41, 70.18, 68.49, 66.23, 21.07;

IR (cm⁻¹): 3011, 2917, 1637, 1496, 1453, 1360, 1215, 1067, 1027, 803, 696, 665, 471, 422;

HRMS-ESI (m/z):[M+Na]⁺ calcd. For [C₆₈H₇₀NaSO₁₀]⁺, 1101.4582. found 1101.4580.

### Example 4: Compound 8

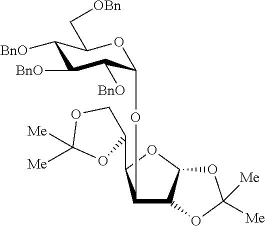

α anomer of 8 was prepared using general Procedure A. Yield: 135 mg, 86%; α:β=1:0; R_{f}=0.45 (hexane/AcOEt=4:1);

¹H NMR (500 MHz, CDCl₃): δ 7.40-7.24 (m, 18H), 7.14 (d, J=6.8 Hz, 2H), 5.89 (d, J=3.6 Hz, 1H), 5.26 (d, J=3.5 Hz, 1H), 4.98 (d, J=10.8 Hz, 1H), 4.83 (dd, J=15.9, 10.7 Hz, 2H), 4.79-4.70 (m, 2H), 4.68 (d, J=3.6 Hz, 1H), 4.63 (d, J=12.1 Hz, 1H), 4.53-4.45 (m, 3H), 4.25 (d, J=2.8 Hz, 1H), 4.15 (dd, J=8.0, 2.8 Hz, 1H), 4.06 (d, J=5.6 Hz, 2H), 3.96 (t, J=9.4 Hz, 1H), 3.84-3.79 (m, 1H), 3.73 (d, J=4.2 Hz, 2H), 3.63 (t, J=9.6 Hz, 1H), 3.58 (dd, J=9.8, 3.5 Hz, 1H), 1.50 (s, 3H, CH₃), 1.43 (s, 3H), 1.26 (s, 3H), 1.26 (s, 3H);

¹³C NMR (125 MHz, CDCl₃): δ 138.63, 138.14, 137.92, 137.81, 128.43, 128.37, 128.04, 127.92, 127.86, 127.73, 127.68, 127.54, 111.76, 109.04, 105.17, 97.92, 83.68, 81.48, 81.17, 80.60, 79.94, 77.64, 75.62, 75.29, 73.54, 73.03, 72.32, 71.17, 68.56, 67.03, 26.98, 26.77, 26.12, 25.46;

IR (cm⁻¹): 2986, 2925, 1497, 1454, 1369, 1254, 1209, 1162, 1064, 1041, 1014, 957, 941, 850, 737, 696, 641, 607, 514, 469;

HRMS-ESI (m/z): [M+Na]f calcd. For [C₄₆H₅₄NaO₁₁]⁺, 805.3558. found 805.3548.

### Example 5: Compound 9

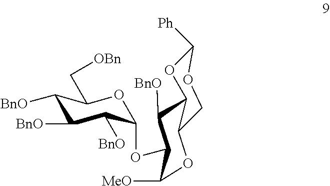

α anomer of 9 was prepared using general Procedure A. Yield: 124 mg, 69%; β:β=10:1; TLC: R_{f}=0.40 (hexane/AcOEt=4:1);

¹H NMR (500 MHz, CDCl₃): δ 7.53-6.98 (m, 30H), 5.55 (s, 1H), 4.93 (d, J=3.5 Hz, 1H), 4.88 (d, J=3.5 Hz, 1H), 4.87-4.75 (m, 5H), 4.70 (d, J=12.0 Hz, 1H), 4.52 (d, J=12.0 Hz, 1H), 4.45 (d, J=11.0 Hz, 1H), 4.32-4.26 (m, 2H), 4.15-4.07 (m, 3H), 3.89-3.83 (m, 2H), 3.73-3.57 (m, 4H), 3.50-3.44 (m, 1H), 3.44 (s, 3H), 3.41-3.36 (m, 1H);

¹³C NMR (126 MHz, CDCl₃): δ 138.82, 138.65, 138.40, 137.97, 137.91, 137.38, 128.89, 128.77, 128.37, 128.27, 128.23, 128.18, 128.01, 127.95, 127.84, 127.76, 127.60, 127.55, 127.43, 125.95, 101.20, 97.20, 94.41, 82.35, 82.10, 79.09, 77.65, 75.66, 75.7, 74.91, 74.26, 73.19, 73.01, 69.89, 69.02, 67.99, 62.25, 54.98;

IR (cm⁻¹): 3031, 2917, 1496, 1451, 1368, 1160, 1100, 1073, 1052, 1025, 998, 969, 902, 850, 781, 730, 694, 643, 611, 574, 539, 491, 459;

HRMS-ESI (m/z): [M+Na]⁺ calcd. For [C₅₅H₅₈NaO₁₁]⁺, 917.3871. found 917.3870.

### Example 6: Compound 10

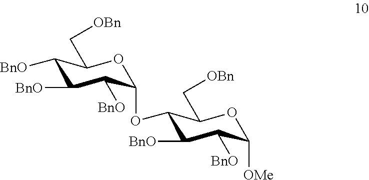

α anomer of 10 was prepared using general Procedure A. Yield: 128 mg, 65%; α:β=4:1 (note, some acceptor co-elutes with product); TLC: R_{f}=0.5 (hexane/AcOEt=4:1);

¹H NMR (500 MHz, CDCl₃): δ 7.37-7.05 (m, 35H), 5.69 (d, J=3.5 Hz, 1H, H-1), 5.03 (d, J=11.5 Hz, 1H), 4.91-4.39 (m, 13H), 4.27 (d, J=12.0 Hz, 1H), 4.11-4.01 (m, 2H), 3.93-3.80 (m, 3H), 3.74-3.69 (m, 1H), 3.67-3.56 (m, 3H), 3.51-3.46 (m, 2H), 3.41-3.39 (m, 1H), 3.37 (s, 3H);

¹³C NMR (126 MHz, CDCl₃): δ 138.95, 138.75, 138.57, 138.48, 138.31, 138.16, 137.97, 128.40, 128.26, 128.19, 128.07, 127.98, 127.89, 127.78, 127.67, 127.59, 127.53, 127.47, 127.33, 127.22, 127.04, 126.73, 97.74, 96.62, 82.01, 80.19, 79.45, 77.63, 75.50, 74.90, 74.75, 73.42, 73.34, 73.20, 73.12, 72.33, 70.95, 69.51, 69.01, 68.12, 55.12;

IR (cm⁻¹): 2918, 1723, 1496, 1453, 1359, 1026, 912, 734, 695, 459;

HRMS-ESI (m/z): [M+Na]⁺ calcd. For [C₆₂H₆₆NaO₁₁]⁺, 1009.4497. found 1009.4498.

### Example 7: Compound 11

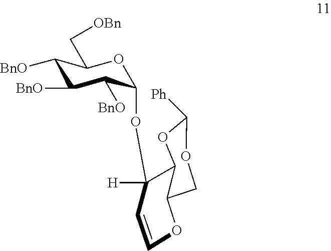

α anomer of 11 was prepared using general Procedure A. Yield: 95 mg, 63%; α:β=7:1; TLC: R_{f}=0.40 (hexane/AcOEt=4:1);

¹H NMR (500 MHz, CDCl₃): δ 7.52-7.45 (m, 2H), 7.43-7.30 (m, 18H), 7.25-7.17 (m, 5H), 6.40 (dd, J=6.0, 1.5 Hz, 1H), 5.59 (s, 1H), 5.49 (d, J=4.0 Hz, 1H), 5.07 (d, J=10.5 Hz, 1H), 4.90 (t, J=6.5 Hz, 2H), 4.80 (dd, J=6.0, 2.0 Hz, 1H), 4.77-4.60 (m, 4H), 4.58-4.51 (m, 2H), 4.41-4.36 (m, 1H), 4.22 (dd, J=8.0, 2.5 Hz, 1H), 4.07 (t, J=9.0 Hz, 1H), 4.03-4.98 (m, 2H), 3.83 (t, J=10.5, Hz, 1H), 3.77 (dd, J=7.0, 3.5 Hz, 1H), 3.70-3.66 (m, 2H), 3.60 (dd, J=9.5, 3.5 Hz, 1H);

¹³C NMR (126 MHz, CDCl₃): δ 144.91, 138.78, 138.12, 137.85, 137.77, 136.99, 129.12, 128.18, 128.10, 127.93, 127.85, 127.82, 127.62, 127.43, 126.11, 102.26, 101.51, 96.38, 81.68, 79.48, 78.95, 77.48, 75.58, 75.04, 73.43, 71.72, 71.28, 70.46, 68.63, 68.38, 68.30;

IR (cm⁻¹): 3029, 2919, 1729, 1637, 1496, 1453, 1374, 1352, 1236, 1095, 1045, 1026, 1010, 955, 916, 877, 836, 736, 694;

HRMS-ESI (m/z): [M+Na]f calcd. For [C₄₇H₄₈NaO₉]⁺, 779.3191. found 779.3190.

### Example 8: Compound 12

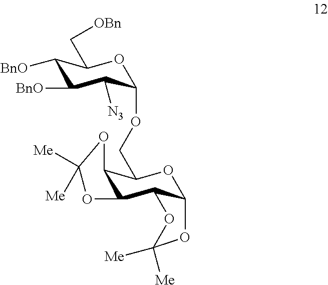

α anomer of 12 was prepared using general Procedure A. Yield:104 mg, 73%; α:β=6:1; TLC: R_{f}=0.45 (hexane/AcOEt=4:1);

¹H NMR (500 MHz, CDCl₃): δ 7.39-7.18 (m, 20H), 5.52 (d, J=5.0 Hz, 1H), 4.99 (d, J=5.0 Hz, 1H, H-1), 4.87 (s, 2H), 4.80 (d, J=11.0 Hz, 1H), 4.70 (s, 1H), 4.65-4.61 (m, 2H), 4.54-4.47 (m, 2H), 4.33-4.30 (m, 2H), 4.02-3.98 (m, 2H), 3.90-3.87 (m, 1H), 3.84-3.70 (m, 4H), 3.67 (dd, J=11.0 Hz, 2.0, 1H), 3.35 (dd, J=11.0, 2.0, 1H), 1.54 (s, 3H), 1.44 (s, 3H), 1.34 (s, 3H), 1.33 (s, 3H);

¹³C NMR (126 MHz, CDCl₃): δ 138.00, 137.81, 128.53, 128.41, 128.37, 127.96, 127.90, 127.78, 127.72, 127.62, 126.94, 109.24, 108.57, 98.24, 96.21, 79.85, 78.19, 75.24, 74.91, 73.47, 70.79, 70.63, 70.61, 70.53, 68.10, 66.80, 66.19, 63.33, 26.08, 25.93, 24.91, 24.34;

IR (cm⁻¹): 2923, 2105, 1496, 1454, 1381, 1309, 1255, 1209, 1152, 1066, 1044, 1001, 918, 889, 862, 735, 696, 510, 459;

HRMS-ESI (m/z) [M+Na]f calcd. For [C₃₉H₄₇NaN₃O₁₀]⁺, 740.3154. found 740.3157.

### Example 9: Compound 13

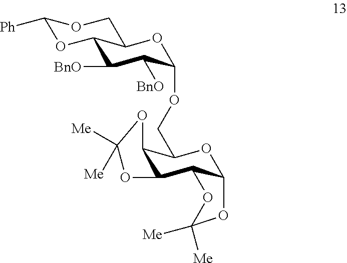

α anomer of 13 was prepared using general Procedure A. Yield: 117 mg, 85%; α:β=20:1; TLC: R_{f}=0.4 (hexane/AcOEt=5:1);

¹H NMR (500 MHz, CDCl₃): δ 7.52-7.50 (m, 2H), 7.40-7.25 (m, 13H), 5.59 (s, 1H), 5.55 (d, J=5.0 Hz, 1H), 4.96-4.75 (m, 5H), 4.62 (dd, J=7.5, 2.0 Hz, 1H), 4.39-4.29 (m, 3H), 4.08-4.04 (m, 2H), 3.93-3.90 (m, 1H), 3.83-3.77 (m, 2H), 3.73 (t, J=10.5 Hz, 1H), 3.64-3.58 (m, 2H), 1.56 (s, 3H), 1.44 (s, 3H), 1.34 (s, 3H), 1.33 (s, 3H);

¹³C NMR (126 MHz, CDCl₃): δ 138.82, 138.26, 137.46, 128.82, 128.48, 128.32, 128.22, 128.16, 127.99, 127.89, 127.73, 127.69, 127.47, 126.00, 109.17, 108.61, 101.14, 98.31, 96.28, 82.06, 79.21, 78.51, 75.21, 72.84, 70.77, 70.61, 68.98, 66.84, 65.86, 62.41, 26.13, 26.02, 24.89, 24.57; IR (cm⁻¹): 2986, 2933, 1496, 1454, 1371, 1255, 1209, 1165, 1087, 1068, 1028, 994, 916, 888, 733, 696, 511, 437;

HRMS-ESI (m/z): [M+Na]⁺ calcd. For [C₃₉H₄₆NaO₁₁]⁺, 713.2932. found 713.2926.

### Example 10: Compound 14

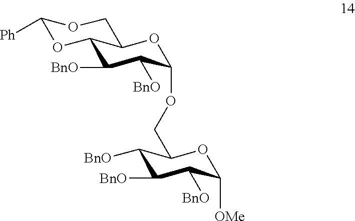

α anomer of 14 was prepared using general Procedure A. Yield: 112 mg, 65%; α:β=18:1; TLC: R_{f}=0.45 (hexane/AcOEt=4:1);

¹H NMR (500 MHz, CDCl₃): δ 7.48-7.23 (m, 30H), 5.55 (s, 1H), 4.98-4.87 (m, 5H), 4.82 (d, J=2.0 Hz, 1H, H-1), 4.80 (m, 1H), 4.74-4.62 (5H), 4.58-4.56 (m, 2H), 4.21 (q, J=5.0 Hz, 1H), 4.02-3.96 (m, 2H), 3.89 (td, J=10.0, 4.5 Hz, 1H), 3.80-3.52 (m, 7H), 3.46-3.42 (m, 2H), 3.34 (s, 3H);

¹³C NMR (125 MHz, CDCl₃): δ 138.81, 138.68, 138.36, 138.14, 137.49, 128.88, 128.41, 128.36, 128.20, 128.01, 127.97, 127.88, 127.73, 127.61, 127.50, 126.05, 101.29, 98.20, 97.97, 82.17, 82.09, 80.06, 79.31, 77.91, 77.72, 75.70, 75.03, 73.35, 72.84, 70.34, 69.08, 66.34, 62.52, 55.19;

IR (cm⁻¹): 3031, 2918, 1497, 1452, 1367, 1327, 1213, 1159, 1088, 1069, 1025, 916, 737, 694, 617, 596, 461;

HRMS-ESI (m/z): [M+Na]⁺ calcd. For [C₅₅H₅₈NaO₁₁]⁺, 917.3871. found 917.3865.

### Example 11: Compound 15

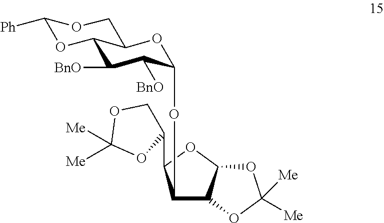

α anomer of 15 was prepared using general Procedure A: Yield:77%:α:β=23:1; Data for α anomer 15: TLC: R_{f}=0.45 (hexane/AcOEt=4:1);

¹H NMR (500 MHz, CDCl₃): δ 7.52-7.50 (m, 2H), 7.42-7.27 (m, 13H), 5.93 (d, J=3.5 Hz, 1H), 5.59 (s, 1H), 5.26 (d, J=3.5 Hz, 1H), 4.94 (d, J=11.0 Hz, 1H), 4.84 (d, J=11.0 Hz, 1H), 4.80 (s, 2H,), 4.59 (d, J=3.5 Hz, 1H), 4.54-4.50 (m, 1H), 4.34 (q, J=4.5 Hz, 1H), 4.25 (d, J=2.5 Hz, 1H), 4.10 (dd, J=8.5, 3.0 Hz, 1H), 4.08-3.99 (m, 3H), 3.89-3.84 (m, 1H), 3.76 (t, J=10.5 Hz, 1H), 3.66 (t, J=10.5 Hz, 1H), 3.60 (dd, J=11.5, 3.5 Hz, 1H), 1.51 (s, 3H), 1.43 (s, 3H), 1.32 (s, 3H), 1.27 (s, 3H);

¹³C NMR (126 MHz, CDCl₃): δ 138.56, 138.05, 137.16, 128.95, 128.34, 128.29, 127.92, 127.71, 127.60, 125.86, 111.93, 109.13, 105.16, 101.2, 98.78, 84.05, 82.20, 81.17, 80.29, 79.20, 78.11, 75.22, 73.68, 72.06, 68.94, 67.12, 63.34, 27.08, 26.81, 26.31, 25.46;

IR (cm⁻¹): 2986, 2934, 1732, 1497, 1454, 1371, 1255, 1212, 1150, 1071, 1017, 913, 841, 734, 696, 678, 655, 507, 457;

HRMS-ESI (m/z): [M+Na]⁺ calcd. For [C₃₉H₄₆NaO₁₁]⁺, 713.2932. found 713.2935.

### Example 12: Compound 16

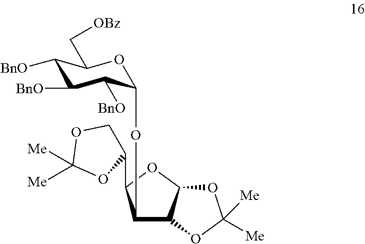

α anomer of 16 was prepared using general Procedure A. Yield: 131 mg, 82%: α:β=1:0; TLC R_{f}=0.45 (hexane/AcOEt=4:1);

¹H NMR (500 MHz, CDCl₃): δ 7.98-7.96 (m, 2H), 7.48 (t, J=6.0 Hz, 1H), 7.36-7.17 (m, 16H), 5.82 (d, J=4.0 Hz, 1H), 5.20 (d, J=3.5 Hz, 1H), 4.92 (d, J=10.5 Hz, 1H), 4.85 (d, J=10.5 Hz, 1H), 4.75 (t, J=11.0 Hz, 1H), 4.64-4.62 (m, 1H), 4.55-4.51 (m, 3H), 4.44-4.38 (m, 2H), 4.20 (d, J=3.0 Hz, 1H), 4.03-3.92 (m, 5H), 3.53-3.48 (m, 2H), 1.41 (s, 3H), 1.32 (s, 3H), 1.16 (s, 3H), 1.14 (s, 3H);

¹³C NMR (126 MHz, CDCl₃): δ 166.30, 138.38, 138.00, 137.48, 133.08, 129.78, 129.58, 128.45, 128.41, 128.35, 128.23, 128.10, 127.97, 127.73, 127.45, 111.87, 109.19, 105.17, 97.55, 84.02, 81.41, 81.22, 80.21, 80.08, 77.82, 75.80, 75.59, 73.09, 72.18, 69.88, 67.16, 63.76, 27.07, 26.82, 26.18, 25.48;

IR (cm⁻¹): 2986, 2932, 1719, 1602, 1497, 1453, 1371, 1335, 1273, 1212, 1145, 1065, 1026, 841, 735, 711, 696, 459, 430;

HRMS-ESI (m/z): [M+Na]⁺ calcd. For [C₄₆H₅₂NaO₁₂]⁺, 819.3351. found 819.3351.

### Example 13: Compound 17

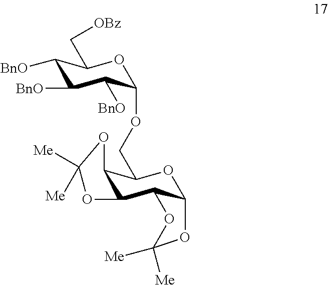

α anomer of 17 was prepared using general Procedure A. Yield:127 mg, 80%; α:β=1:0; TLC: R_{f}=0.40 (hexane/AcOEt=5:1);

¹H NMR (500 MHz, CDCl₃): δ 8.03-8.02 (m, 2H), 7.56-7.22 (18H), 5.55 (d, J=5.0 Hz, 1H), 5.08 (d, J=10.5 Hz, 1H), 5.01 (d, J=3.5 Hz, 1H), 4.92 (d, J=11.0 Hz, 1H), 4.85 (d, J=11.0 Hz, 1H), 4.79 (d, J=11.0 Hz, 1H), 4.73 (d, J=11.0 Hz, 1H), 4.64-4.59 (m, 2H), 4.55 (d, J=2.5 Hz, 1H), 4.33-4.32 (m, 2H), 4.14-4.07 (m, 3H), 3.86-3.77 (m, 2H), 3.68 (m, 2H), 1.56 (s, 3H), 1.45 (s, 3H), 1.34 (s, 3H), 1.32 (s, 3H);

¹³C NMR (126 MHz, CDCl₃): δ 166.19, 138.58, 138.17, 137.83, 132.94, 129.89, 129.64, 128.39, 128.32, 128.06, 127.99, 127.80, 127.74, 127.65, 109.20, 108.56, 96.72, 96.27, 81.89, 79.96, 77.47, 75.80, 75.00, 72.35, 70.87, 70.62, 70.53, 68.77, 66.55, 65.78, 63.45, 26.10, 26.01, 24.88, 24.59;

IR (cm⁻¹): 2917, 1719, 1602, 1497, 1453, 1381, 1337, 1273, 1207, 1164, 1066, 1026, 998, 918, 888, 735, 711, 696, 672, 512, 486, 463, 420;

HRMS-ESI (m/z): [M+Na]⁺ calcd. For [C₄₆H₅₂NaO₁₂]⁺, 819.3351. found 819.3352.

### Example 14: Compound 18

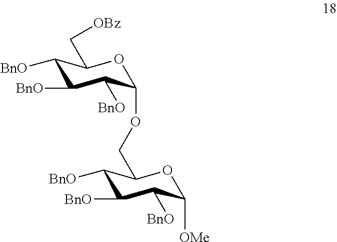

α anomer of 18 was prepared using general Procedure A. Yield: 132 mg, 66%; α:β=12:1; TLC: R_{f}=0.40 (hexane/AcOEt=4:1);

¹H NMR (500 MHz, CDCl₃): δ 7.90-7.88 (m, 2H), 7.47-7.44 (m, 1H), 7.33-7.14 (m, 32H), 4.90-4.81 (m, 5H), 4.73 (dd, J=10.5, 4.5 Hz, 1H), 4.63-4.40 (m, 8H), 4.31 (dd, J=12.0, 4.5 Hz, 1H), 3.95-3.88 (m, 3H), 3.74-3.68 (m, 2H), 3.63-3.61 (m, 1H), 3.54-3.50 (m, 2H), 3.47 (dd, J=10.0, 4.0 Hz, 1H), 3.33 (dd, J=9.5, 3.5 Hz, 1H), 3.27 (s, 3H);

¹³C NMR (126 MHz, CDCl₃): δ 166.16, 138.79, 138.49, 138.30, 138.12, 137.96, 132.99, 129.95, 129.61, 128.39, 128.17, 127.96, 127.90, 127.82, 127.76, 127.71, 127.67, 127.57, 97.88, 96.87, 82.07, 81.67, 80.15, 80.12, 77.81, 77.52, 75.71, 75.00, 73.31, 72.41, 70.34, 68.85, 65.98, 63.41, 55.15;

IR (cm⁻¹): 3030, 2916, 1719, 1602, 1496, 1452, 1358, 1272, 1159, 1088, 1068, 1025, 912, 733, 711, 695, 611, 531, 460;

HRMS-ESI (m/z): [M+Na]⁺ calcd. For [C₆₂H₆₄NaO₁₂]⁺, 1023.4290. found 1023.4288.

### Example 15: Compound 19

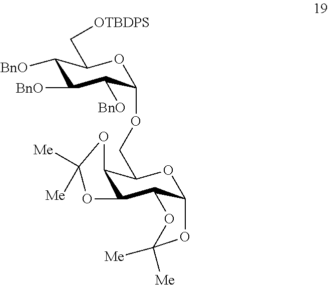

α anomer of 19 was prepared using general Procedure A. Yield: 115 mg: 62%; α:β=7:1; TLC R_{f}=0.45 (hexane/AcOEt=5:1);

¹H NMR (500 MHz, CDCl₃): δ 7.74-7.70 (m, 4H), 7.45-7.40 (m, 4H), 7.39-7.29 (m, 16H), 7.23-7.20 (m, 1H), 5.54 (d, J=5.0 Hz, 1H), 5.09 (d, J=3.5 Hz, 1H), 5.02 (d, J=10.5 Hz, 1H), 4.94 (d, J=10.5 Hz, 1H), 4.86-4.83 (m, 2H), 4.75-4.69 (m, 2H), 4.63 (dd, J=8.0, 2.5 Hz, 1H), 4.38 (dd, J=8.0, 2.0 Hz, 1H), 4.33 (q, J=2.5 Hz, 1H), 4.08-4.04 (m, 2H), 3.99-3.90 (m, 2H), 3.83-3.70 (m, 3H), 3.63 (dd, J=9.5, 3.5 Hz, 1H), 1.54 (s, 3H), 1.49 (s, 3H), 1.34 (s, 6H), 1.08 (s, 9H);

¹³C NMR (126 MHz, CDCl₃): δ 138.85, 138.48, 138.37, 135.89, 135.80, 135.59, 133.67, 133.23, 129.54, 129.50, 128.34, 128.31, 128.21, 128.09, 127.83, 127.69, 127.62, 127.57, 127.49, 109.13, 108.49, 96.28, 82.04, 80.21, 77.58, 75.9, 75.79, 75.07, 72.13, 71.48, 70.77, 70.64, 70.62, 65.46, 65.32, 62.64, 26.80, 26.11, 26.05, 24.88, 24.60, 19.30;

IR (cm⁻¹): 2930, 1496, 1454, 1428, 1381, 1255, 1210, 1162, 1067, 998, 918, 823, 735, 696, 648, 612, 504, 466;

HRMS-ESI (m/z): [M+Na]f calcd. For [C₅₅H₆₆NaSiO₁₁]⁺, 953.4267. found 953.4278.

### Example 16: Compound 20

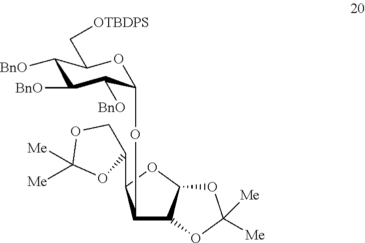

α anomer 20 was prepared by using General Procedure A: Yield: 116 mg, 63%: α:β=1:0; TLC: R_{f}=0.45 (hexane/AcOEt=4:1);

¹H NMR (500 MHz, CDCl₃): δ 7.73-7.70 (m, 4H), 7.43-7.27 (m, 19H), 7.15-7.13 (m, 2H), 5.84 (d, J=3.5 Hz, 1H, H-1), 5.26 (d, J=3.0 Hz, 1H), 4.97 (d, J=11.0 Hz, 1H), 4.90 (d, J=10.5 Hz, 1H), 4.84-4.80 (m, 2H), 4.74 (d, J=12.0 Hz, 1H), 4.61-4.58 (m, 2H), 4.52-4.51 (m, 1H), 4.25 (d, J=3.0 Hz, 1H), 4.13 (dd, J=8.0, 2.5 Hz, 1H), 4.08-4.01 (m, 2H), 3.99 (t, J=9.0 Hz, 1H), 3.91 (d, J=3.0 Hz, 1H), 3.76-3.74 (m, 1H), 3.67 (t, J=9.0 Hz, 1H), 3.57 (dd, J=10.0, 3.5 Hz, 1H), 1.47 (s, 3H), 1.45 (s, 3H), 1.27 (s, 3H), 1.20 (s, 3H), 1.07 (s, 9H);

¹³C NMR (126 MHz, CDCl₃): δ 138.59 138.27, 137.97, 135.80, 135.66, 133.43, 133.17, 129.65, 129.60, 128.45, 128.41, 128.38, 128.04, 128.02, 127.82, 127.69, 127.64, 127.59, 127.41, 111.82, 105.13, 97.78, 83.83, 81.49, 81.22, 80.43, 80.27, 77.69, 75.77, 75.40, 73.16, 72.31, 67.07, 62.91, 29.68, 27.06, 26.84, 26.26, 25.47, 19.27;

IR (cm⁻¹): 2930, 1497, 1454, 1427, 1371, 1255, 1213, 1145, 1066, 1036, 957, 882, 842, 823, 736, 696, 613, 503, 489;

HRMS-ESI (m/z): [M+Na]⁺ calcd. For [C₅₅H₆₆NaSiO₁₁]⁺, 953.4267. found 953.4277.

### Example 17: Compound 21

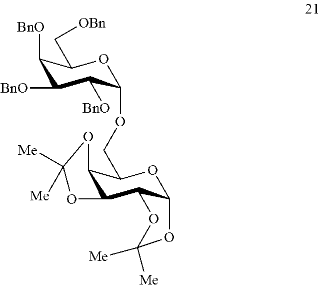

α anomer 21 was prepared by using General Procedure B: Yield: 137 mg, 88%; α:β=20:1; TLC: R_{f}=0.45 (hexane/AcOEt=4:1);

¹H NMR (500 MHz, CDCl₃): δ 7.38 (ddd, J=7.9, 3.5, 1.5 Hz, 3H), 7.35-7.24 (m, 17H), 5.51 (d, J=5.0 Hz, 1H), 5.01 (d, J=3.7 Hz, 1H), 4.94 (d, J=11.5 Hz, 1H), 4.83 (d, J=11.7 Hz, 1H), 4.79-4.70 (m, 3H), 4.61-4.54 (m, 2H), 4.48 (d, J=11.8 Hz, 1H), 4.42 (d, J=11.8 Hz, 1H), 4.34-4.27 (m, 2H), 4.09-3.99 (m, 4H), 3.96 (dd, J=10.1, 2.8 Hz, 1H), 3.79 (dd, J=10.5, 6.4 Hz, 1H), 3.74 (dd, J=10.5, 7.1 Hz, 1H), 3.58 (dd, J=9.2, 7.5 Hz, 1H), 3.52 (dd, J=9.2, 5.7 Hz, 1H), 1.52 (s, 3H), 1.43 (s, 3H), 1.33 (s, 3H), 1.30 (s, 3H);

¹³C NMR (126 MHz, CDCl₃): δ 138.91, 138.73, 138.05, 128.35, 128.28, 128.20, 128.16, 127.79, 127.70, 127.63, 127.46, 127.44, 127.39, 109.15, 108.48, 97.53 96.30, 78.96, 76.39, 74.91, 74.74, 73.36, 73.02, 72.63, 70.85, 70.61, 69.12, 68.65, 66.30, 65.78, 26.13, 26.02, 24.91, 24.57;

IR (cm⁻¹): 2917, 1496, 1453, 1370, 1308, 1254, 1209, 1165, 1095, 1066, 998, 917, 889, 865, 734, 695, 609, 511, 461;

HRMS-ESI (m/z): [M+Na]⁺ calcd. For [C₄₆H₅₄NaO₁₁]⁺, 805.3558. found 805.3549.

### Example 18: Compound 22

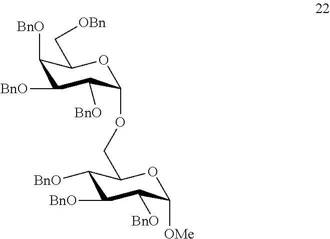

α anomer 22 was prepared by using General Procedure B: Yield: 146 mg, 74%; α:β=1:0; TLC: R_{f}=0.40 (hexane/AcOEt=4:1);

¹H NMR (500 MHz, CDCl₃): δ 7.35-7.22 (m, 35H), 4.98 (d, J=3.5 Hz, 1H), 4.94 (t, J=11.5 Hz, 2H), 4.84 (d, J=11.0 Hz, 1H), 4.81-4.77 (m, 2H), 4.74-4.68 (m, 4H), 4.58 (d, J=11.0 Hz, 2H), 4.55-4.52 (m, 2H), 4.42 (d, J=11.5 Hz, 1H), 4.35 (d, J=11.5 Hz, 1H), 4.02 (dd, J=9.5, 3.5 Hz, 1H), 3.98-3.88 (m, 4H), 3.81-3.74 (m, 2H), 3.72 (d, J=11.5 Hz, 1H), 3.58 (t, J=9.5 Hz, 1H), 3.51-3.47 (m, 2H), 3.41 (dd, J=9.5, 3.5 Hz, 1H), 3.29 (s, 3H);

¹³C NMR (126 MHz, CDCl₃): δ 138.86, 138.75, 138.70, 138.40, 138.20, 138.04, 128.38, 128.33, 128.28, 128.22, 128.19, 127.95, 127.79, 127.68, 127.63, 127.49, 127.40, 127.34, 97.91, 97.86, 82.06, 80.15, 78.24, 77.98, 76.52, 75.67, 75.08, 74.98, 74.73, 73.32, 72.79, 72.51, 70.28, 69.36, 68.91, 66.39, 55.01;

IR (cm⁻¹): 3030, 2917, 1721, 1496, 1453, 1355, 1278, 1208, 1132, 1091, 1025, 911, 732, 694, 607, 458;

HRMS-ESI (m/z): [M+Na]⁺ calcd. For [C₆₂H₆₆NaO₁₁]⁺, 1009.4497. found 1009.4494.

### Example 19: Compound 23

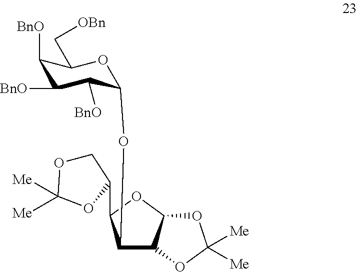

α anomer 23 was prepared by using General Procedure B: Yield: 127 mg, 81%; α:β=1:0; R_{f}=0.40 (hexane/AcOEt=4:1);

¹H NMR (500 MHz, CDCl₃): δ 7.40-7.24 (m, 20H), 5.87 (d, J=3.5 Hz, 1H), 5.21 (d, J=4.0 Hz, 1H), 4.96 (d, J=11.5 Hz, 1H), 4.84 (d, J=11.5 Hz, 1H), 4.79 (d, J=11.5 Hz, 1H), 4.76-4.70 (m, 3H), 4.57 (d, J=11.5 Hz, 1H), 4.53-4.44 (m, 2H), 4.42 (d, J=12.0 Hz, 1H), 4.18 (d, J=5.0 Hz, 2H), 4.04 (tdd, J=13.5, 8.5, 4.5 Hz, 3H), 3.94 (d, J=4.0 Hz, 2H), 3.87 (dd, J=10.0, 2.5 Hz, 1H), 3.58 (dd, J=9.5, 6.5 Hz, 1H), 3.47 (dd, J=9.5, 5.5 Hz, 1H), 1.48 (s, 3H), 1.42 (s, 3H), 1.24 (s, 3H), 1.19 (s, 3H);

¹³C NMR (126 MHz, CDCl₃): δ 138.23, 137.91, 137.81, 137.16, 128.64, 128.58, 128.55, 128.42, 128.30, 128.16, 127.82, 127.75, 127.65, 127.55, 111.79, 105.64, 101.30, 86.00, 82.82, 80.24, 79.07, 75.62, 75.07, 74.86, 74.67, 74.37, 73.54, 72.41, 70.86, 69.83, 69.55, 64.70, 26.79, 26.06;

IR (cm⁻¹): 2915, 1496, 1453, 1355, 1276, 1209, 1132, 1090, 1049, 1025, 912, 781, 733, 694, 633, 620, 605, 508, 455, 430;

HRMS-ESI (m/z): [M+Na]⁺ calcd. For [C₄₆H₅₄NaO₁₁]⁺, 805.3558. found 805.3548.

### Example 20: Compound 24

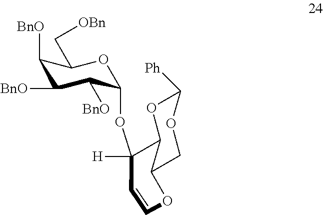

α anomer 24 was prepared by using General Procedure B: Yield: 104 mg, 69%; α:β=1:0; TLC: R_{f}=0.45 (hexane/AcOEt=4:1);

¹H NMR (500 MHz, CDCl₃): δ 7.42-7.11 (m, 25H), 6.33 (dd, J=6.0, 1.0 Hz, 1H), 5.53 (s, 1H), 5.42 (d, J=3.5 Hz, 1H), 5.29 (s, 1H), 4.93 (d, J=11.5 Hz, 1H), 4.86 (J=11.5 Hz, 1H), 4.76-4.62 (m, 4H), 4.55 (d, J=11.5 Hz, 1H), 4.44 (d, J=11.5 Hz, 1H), 4.37-4.30 (m, 2H), 4.23-4.20 (m, 1H), 4.14-3.89 (m, 6H), 3.78 (t, J=10.0 Hz, 1H), 3.56-3.51 (m, 1H);

¹³C NMR (126 MHz, CDCl₃): δ 144.97, 138.91, 138.63, 138.45, 137.96, 137.12, 129.09, 128.37, 128.30, 128.18, 127.89, 127.78, 127.71, 127.52, 127.46, 127.38, 127.33, 126.10, 102.52, 101.35, 97.09, 79.68, 79.42, 78.91, 78.68, 75.92, 75.08, 74.76, 74.48, 73.50, 73.23, 72.28, 71.52, 69.67, 69.17, 68.81, 68.36;

IR (cm⁻¹): 3029, 2919, 1729, 1637, 1496, 1453, 1374, 1352, 1236, 1095, 1045, 1026, 1010, 955, 916, 877, 836, 736, 694;

HRMS-ESI (m/z): [M+Na]⁺ calcd. For [C₄₇H₄₈NaO₉]⁺, 779.3191. found 779.3219.

### Example 21: Compound 25

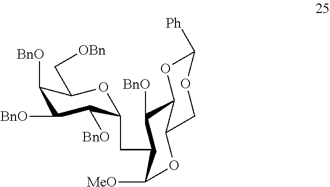

α anomer 25 was prepared by using General Procedure B: Yield: 130 mg, 73%, α:β=1:0; TLC: R_{f}=0.35 (hexane/AcOEt=4:1);

¹H NMR (500 MHz, CDCl₃): δ 7.59-7.08 (m, 30H, ArH), 5.52 (s, 1H), 4.96 (d, J=3.2 Hz, 1H), 4.93-4.77 (m, 5H), 4.73-4.66 (m, 3H), 4.52 (d, J=11.4 Hz, 1H), 4.32-4.23 (m, 4H), 4.09 (t, J=9.4 Hz, 1H), 4.03 (dd, J=10.5, 2.9 Hz, 1H), 3.95 (dd, J=10.1, 2.4 Hz, 1H), 3.88-3.75 (m, 3H), 3.70 (t, J=10.1 Hz, 1H), 3.54 (t, J=9.5 Hz, 1H), 3.48 (dd, J=9.5, 6.5 Hz, 1H), 3.42 (s, 3H), 3.34 (dd, J=9.5, 6.8 Hz, 1H);

¹³C NMR (126 MHz, CDCl₃): δ 138.86, 138.68, 138.42, 137.40, 128.87, 128.35, 128.25, 128.15, 128.09, 127.88, 127.75, 127.56, 127.48, 127.44, 127.38, 125.98, 101.17, 97.36, 94.88, 82.39, 78.83, 75.94, 75.34, 74.98, 74.72, 73.94, 73.08, 72.89, 72.78, 69.04, 68.95, 68.84, 62.29, 55.06;

IR (cm⁻¹): 3031, 2917, 1496, 1451, 1368, 1347, 1329, 1213, 1160, 1131, 1100, 1073, 1052, 1025, 998, 969, 902, 850, 781, 694, 611, 459;

HRMS-ESI (m/z): [M+Na]⁺ calcd. For [C₅₅H₅₈NaO₁₁]⁺, 917.3871. found 917.3869.

### Example 22: Compound 26

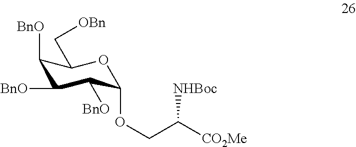

α anomer 26 was prepared by using General Procedure B: Yield: 109 mg, 74%, α:β=1:0; TLC: R_{f}=0.3 (hexane/AcOEt=4:1);

¹H NMR (500 MHz, CDCl₃): δ 7.33-7.27 (m, 20H), 5.68 (d, J=8.5 Hz, 1H), 4.84 (d, J=11.0 Hz, 1H), 4.73-4.62 (m, 4H), 4.54 (d, J=12.0 Hz, 1H), 4.48-4.42 (m, 2H), 4.34-4.32 (m, 2H), 4.03 (dd, J=11.0, 3.5 Hz, 1H), 3.94 (dd, J=10.5, 4.0 Hz, 1H), 3.88-3.85 (m, 2H), 3.78 (dd, J=10.5, 3.0 Hz, 1H), 3.71 (dd, J=11.0, 2.5 Hz, 1H), 3.56 (s, 3H), 3.46-3.45 (m, 2H), 1.34 (s, 9H);

¹³C NMR (126 MHz, CDCl₃): δ 170.87, 155.55, 138.65, 138.51, 138.49, 137.88, 128.33, 128.28, 128.18, 127.8, 127.73, 127.64, 127.57, 127.4, 127.42, 127.39, 99.23, 79.82, 78.61, 76.34, 74.72, 74.67, 73.41, 73.12, 72.97, 70.26, 69.71, 68.60, 54.13, 52.30, 28.27;

IR (cm⁻¹): 3029, 2928, 1748, 1713, 1496, 1453, 1391, 1365, 1348, 1300, 1246, 1209, 1157, 1093, 1054, 1027, 911, 734, 696, 601, 460;

HRMS-ESI (m/z): [M+H]⁺ calcd. For [C₄₃H₅₂NO₁₀]⁺, 742.3591. found 742.3588.

### Example 23: Compound 27

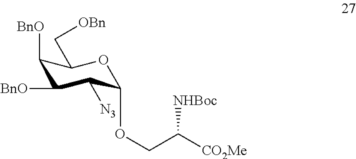

α anomer 27 was prepared by using General Procedure B: Yield: 62%, α:β=1:0; TLC: R_{f}=0.45 (hexane/AcOEt=4:1);

¹H NMR (500 MHz, CDCl₃): δ 7.31-7.17 (m, 35H), 5.76 (d, J=3.5 Hz, 1H), 4.97 (d, J=11.5 Hz, 1H), 4.86 (d, J=11.5 Hz, 1H), 4.81 (d, J=11.5 Hz, 1H), 4.71-4.65 (m, 4H), 4.60 (d, J=11.5 Hz, 1H), 4.57-4.51 (m, 4H), 4.41 (d, J=11.5 Hz, 1H), 4.29 (d, J=11.5 Hz, 1H), 4.22 (d, J=11.5 Hz, 1H), 4.07 (t, J=9.5 Hz, 1H), 4.00-3.96 (m, 2H), 3.93-3.84 (m, 3H), 3.81 (dd, J=10.0, 2.5 Hz, 1H), 3.70 (d, J=11.0, 3.5 Hz, 1H), 3.64 (dd, J=11.0, 2.5 Hz, 1H), 3.55 (dd, J=9.5, 3.5 Hz, 1H), 3.49-3.39 (m, 2H), 3.37 (s, 3H);

¹³C NMR (126 MHz, CDCl₃): δ 138.94, 128.57, 138.32, 138.23, 137.96, 128.35, 128.28, 128.22, 128.16, 127.83, 127.72, 127.64, 127.53, 127.46, 127.40, 127.32, 126.98, 126.68, 97.68, 97.43, 81.97, 80.12, 79.12, 75.59, 74.72, 74.60, 74.29, 73.74, 73.37, 73.32, 73.02, 72.72, 69.82, 69.43, 69.39, 68.64, 55.04;

IR (cm⁻¹): 3029, 2918, 1723, 1496, 1453, 1361, 1273, 1207, 1093, 1040, 1027, 911, 732, 694, 607, 548, 461;

HRMS-ESI (m/z): [M+Na]⁺ calcd. For [C₆₂H₆₆NaO₁₁]⁺, 1009.4497. found 1009.4498.

### Example 24: Compound 28

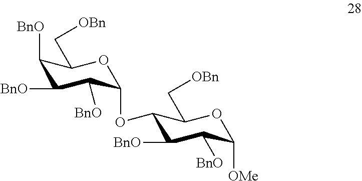

α anomer 28 was prepared by using General Procedure B: Yield: 112 mg, 83%; α:β=1:0; TLC: R_{f}=0.45 (hexane/AcOEt=4:1);

¹H NMR (500 MHz, CDCl₃): δ 7.35-7.24 (m, 15H), 5.56 (d, J=8.5 Hz, 1H), 4.86 (dd, J=7.5, 4.0 Hz, 1H), 4.74 (d, J=11.5 Hz, 1H), 4.67 (d, J=11.5 Hz, 1H), 4.54-4.51 (m, 3H), 4.44 (d, J=12.0 Hz, 1H), 4.06-4.02 (m, 2H), 3.96 (t, J=6.5 Hz, 1H), 3.93-3.88 (m, 3H), 3.82 (dd, J=11.0, 3.5 Hz, 1H), 3.76 (s, 3H), 3.61-3.54 (m, 2H), 1.45 (s, 9H);

¹³C NMR (126 MHz, CDCl₃): δ 170.60, 155.42, 138.15, 137.73, 137.42, 128.50, 128.42, 128.27, 128.09, 127.91, 127.82, 127.72, 99.46, 80.07, 74.82, 73.51, 73.06, 72.11, 69.90, 69.78, 68.35, 59.54, 54.03, 52.59, 28.28;

IR (cm⁻¹): 3367, 2930, 2162, 2151, 2108, 1747, 1713, 1496, 1454, 1365, 1348, 1248, 1210, 1157, 1095, 1051, 984, 735, 696, 654, 461;

HRMS-ESI (m/z): [M+Na]⁺ calcd. For [C₃₆H₄₄NaN₄O₉]⁺, 699.3001. found 699.2992.

### Example 25: Compound 29

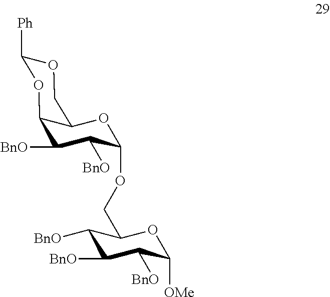

α anomer 29 was prepared by using General Procedure B: Yield: 134 mg, 75%; α:β=20:1; TLC: R_{f}=0.35 (hexane/AcOEt=4:1);

¹H NMR (500 MHz, CDCl₃): δ 7.53-7.51 (m, 2H), 7.39-7.21 (m, 28H), 5.46 (s, 1H), 5.06 (d, J=3.5 Hz, 1H), 4.99 (d, J=10.5 Hz, 1H), 4.90 (d, J=11.5 Hz, 1H), 4.82-4.68 (m, 7H), 4.61-4.53 (m, 3H), 4.13-4.10 (m, 2H), 4.07 (dd, J=10.0, 3.5 Hz, 1H), 3.99 (t, J=9.5 Hz, 1H), 3.95 (dd, J=10.5, 7.5 Hz, 1H), 3.88 (d, J=10.0, 3.5 Hz, 1H), 3.78-3.69 (m, 3H), 3.58 (t, J=10.0, 3.5 Hz, 1H), 3.49 (s, 1H), 3.45 (dd, J=9.5, 3.5 Hz, 1H), 3.3 (s, 3H);

¹³C NMR (126 MHz, CDCl₃): δ 138.85, 138.78, 138.66, 138.50, 138.15, 137.83, 128.82, 128.39, 128.34, 128.25, 128.06, 127.96, 127.82, 127.73, 127.56, 127.50, 127.48, 127.37, 126.34, 101.03, 98.35, 97.86, 82.09, 80.12, 77.99, 75.66, 75.61, 74.90, 74.80, 73.32, 72.81, 71.84, 70.11, 69.34, 66.43, 62.58, 54.97;

IR (cm⁻¹): 3030, 2917, 1496, 1453, 1358, 1249, 1211, 1130, 1069, 1049, 1026, 996, 917, 831, 795, 734, 694, 614, 558, 505, 461;

HRMS-ESI (m/z): [M+Na]⁺ calcd. For [C₅₅H₅₈NaO₁₁]⁺, 917.3871. found 917.3867.

### Example 26: Compound 30

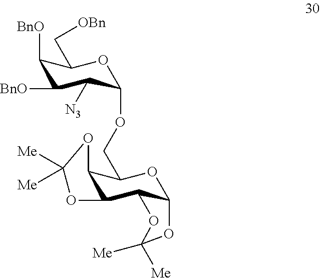

α anomer 30 was prepared by using General Procedure B: Yield: 116 mg, 81%, α:β=1:0; TLC: R_{f}=0.40 (hexane/AcOEt=4:1);

¹H NMR (500 MHz, CDCl₃): δ 7.36-7.28 (m, 15H), 5.52 (d, J=5.0 Hz, 1H), 4.97 (d, J=4.0 Hz, 1H), 4.88 (d, J=11.5 Hz, 1H), 4.74 (d, J=11.5 Hz, 1H), 4.68 (d, J=11.0 Hz, 1H), 4.61 (dd, J=7.5, 2.5 Hz, 1H), 4.56-4.43 (m, 3H), 4.32-4.29 (m, 2H), 4.10-3.99 (m, 4H), 3.85-3.81 (m, 2H), 3.70 (dd, J=10, 5.5 Hz, 1H), 3.62 (t, J=9.5 Hz, 1H), 3.55 (dd, J=9.0, 5.5 Hz, 1H), 1.54 (s, 3H), 1.42 (s, 3H), 1.34 (s, 3H), 1.33 (s, 3H);

¹³C NMR (126 MHz, CDCl₃): δ 138.32, 137.88, 137.60, 128.75, 128.44, 128.36, 128.21, 128.07, 127.80, 127.70, 127.61, 109.24, 108.53, 98.33, 96.22, 74.77, 73.32, 72.08, 70.93, 70.56, 69.29, 68.33, 66.87, 66.45, 59.67, 26.03, 25.90, 24.93, 24.34;

IR (cm⁻¹): 2923, 2107, 1725, 1496, 1454, 1381, 1309, 1255, 1209, 1165, 1115, 1095, 1066, 1003, 918, 889, 865, 734, 696, 650, 599, 506, 462, 429;

HRMS-ESI (m/z): [M+Na]⁺ calcd. For [C₃₉H₄₇NaN₃O₁₀]⁺, 740.3154. found 740.3143.

### Example 27: Compound 31

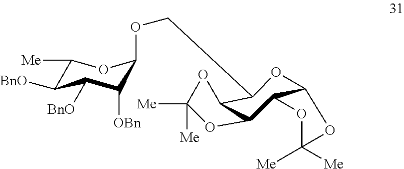

α anomer 31 was prepared by using General Procedure B: Yield: 111 mg, 82%, α:β=1:0; TLC: R_{f}=0.45 (hexane/AcOEt=5:1);

¹H NMR (500 MHz, CDCl₃): δ 7.43-7.28 (m, 15H), 5.55 (d, J=5.0 Hz, 1H), 4.96 (d, J=11 Hz, 1H), 4.93 (d, J=1.5 Hz, 1H), 4.77 (s, 2H), 4.69-4.60 (m, 4H), 4.33 (q, J=2.5 Hz, 1H), 4.19 (dd, J=7.5, 1.5 Hz, 1H), 3.95-3.78 (m, 5H), 3.66 (t, J=9.5 Hz, 1H), 3.61 (dd, J=7.0, 3.5 Hz, 1H), 1.55 (s, 3H), 1.47 (s, 3H), 1.36 (s, 6H), 1.35 (d, J=6.0 Hz, 3H);

¹³C NMR (126 MHz, CDCl₃): δ 138.65, 138.50, 138.33, 128.19, 127.96, 127.80, 127.52, 127.46, 127.35, 109.18, 108.43, 97.90, 96.14, 79.85, 75.11, 74.63, 72.48, 71.82, 71.04, 70.50, 70.43, 67.96, 67.14, 65.81, 26.05, 25.88, 24.87, 24.30;

IR (cm⁻¹): 2986, 2932, 1496, 1454, 1381, 1254, 1209, 1167, 1063, 1027, 1000, 917, 900, 801, 734, 696, 647, 511, 457;

HRMS-ESI (m/z): [M+Na]⁺ calcd. For [C₃₉H₄₈NaO₁₀]⁺, 699.3140. found 699.3129.

### Example 28: Compound 32

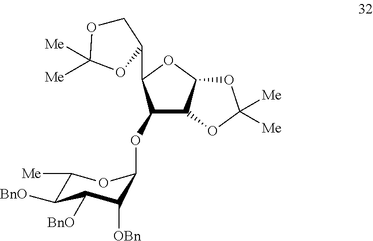

α anomer 32 was prepared by using General Procedure B: Yield: 105 mg, 78%, α:β=1:0; TLC: R_{f}=0.45 (hexane/AcOEt=5:1);

¹H NMR (500 MHz, CDCl₃): δ 7.40-7.27 (m, 15H), 5.77 (d, J=4.0 Hz, 1H), 4.93 (d, J=11.0 Hz, 1H), 4.86 (d, J=12.0 Hz, 1H), 4.76 (d, J=1.5 Hz, 1H), 4.67-4.64 (m, 4H), 4.28 (d, J=3.5 Hz, 1H), 4.19-4.15 (m, 2H), 4.09-4.06 (m, 2H), 3.99-3.96 (m, 1H), 3.90 (dd, J=8.5, 6.0 Hz, 1H), 3.75 (dd, J=9.0, 3.0 Hz, 1H), 3.69 (m, 1H), 3.60 (t, J=10.0 Hz, 1H), 1.49 (s, 3H), 1.38 (s, 3H), 1.31 (s, H), 1.28 (d, J=6.0 Hz, 3H), 1.28 (s, 3H);

¹³C NMR (126 MHz, CDCl₃): δ 138.94, 138.37, 138.18, 128.37, 128.32, 128.18, 128.16, 127.78, 127.71, 127.58, 127.49, 127.35, 111.87, 109.16, 105.14, 95.86, 81.68, 80.90, 80.22, 79.78, 76.61, 75.20, 74.82, 73.30, 72.48, 71.89, 68.50, 67.76, 26.70, 26.15, 25.19, 17.62;

IR (cm⁻¹): 3029, 2916, 1496, 1453, 1371, 1355, 1208, 1131, 1091, 1072, 1025, 911, 847, 733, 695, 532, 459;

HRMS-ESI (m/z): [M+Na]f calcd. For [C₃₉H₄₈NaO₁₀]⁺, 699.3140. found 699.3124.

### Example 29: Compound 33

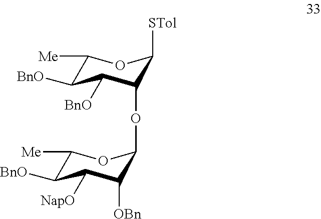

α anomer of 33 was prepared using General Procedure B: 141 mg, 78%, α:β=20:1; TLC: R_{f}=0.45 (hexane/AcOEt=5:1);

¹H NMR (500 MHz, CDCl₃): δ 7.69-7.60 (m, 4H), 7.34-7.30 (m, 2H), 7.25-7.09 (m, 23H), 6.98 (d, J=10.0 Hz, 2H), 5.23 (d, J=1.0 Hz, 1H), 5.00 (d, J=1.0 Hz, 1H), 4.86 (m, 1H), 4.69-4.62 (m, 3H), 4.55-4.51 (m, 4H), 4.46 (t, J=12.5 Hz, 1H), 4.37-4.35 (m, 1H), 4.13 (s, 1H), 3.98-3.94 (m, 1H), 3.82 (dd, J=9.5, 3.0 Hz, 1H), 3.74-3.70 (m, 2H), 3.67-3.64 (m, 1H), 3.54 (t, J=9.0 Hz, 1H), 3.17 (t, J=9.0 Hz, 1H), 2.2 (s, 3H), 1.17 (d, J=6.0 Hz, 3H), 1.07 (d, J=6.0 Hz, 3H);

¹³C NMR (126 MHz, CDCl₃): δ 138.51, 138.34, 138.22, 137.91, 137.40, 136.02, 133.20, 132.86, 131.71, 130.62, 129.78, 128.48, 128.35, 128.31, 128.19, 127.91, 127.68, 127.72, 127.60, 127.57, 127.51, 126.31, 125.97, 125.76, 99.41, 87.61, 80.58, 80.30, 80.10, 79.05, 75.91, 75.29, 75.19, 75.00, 74.87, 72.70, 72.46, 72.20, 69.16, 68.81, 68.57, 68.24, 21.03, 17.91, 17.71;

IR (cm⁻¹): 3029, 2910, 1731, 1602, 1494, 1453, 1364, 1282, 1208, 1086, 1060, 1026, 911, 843, 810, 733, 696, 620, 474;

HRMS-ESI (m/z): [M+Na]⁺ calcd. For [C₅₈H₆₀NaSO₈]⁺, 939.3901. found 939.3904.

### Example 30: Compound 34

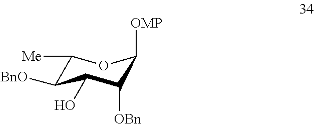

α anomer of 34 was prepared according to procedure well-known in the art (see P. R. Verma et al., Carbohydrate Res., 2010, 345, 432-436). Yield: 1.35 g, 68%; TLC: R_{f}=0.45 (hexane/AcOEt=1:1);

¹H NMR (500 MHz, CDCl₃): δ 7.38-7.27 (m, 5H), 6.99-6.97 (m, 2H), 6.85-6.82 (m, 2H), 5.40 (s, 1H), 4.79-4.74 (m, 2H), 4.14-4.10 (m, 2H), 3.90-3.86 (dq, 1H, J=9.4, 6.1 Hz), 3.78 (s, 3H), 3.43 (t, J=9.4 Hz, 1H), 2.50 (br s, 1H), 2.38 (br s, 1H), 1.33 (d, J=6.1 Hz, 3H);

¹³C NMR (126 MHz, CDCl₃): δ 154.90, 150.19, 138.15, 128.67, 128.07, 127.95, 117.54, 114.59, 98.07, 81.58, 75.10, 71.26, 71.04, 67.86, 55.63, 18.02;

IR (cm⁻¹): 3011, 2917, 1637, 1496, 1453, 1360, 1215, 1067, 1027, 803, 696, 665, 471, 422.

### Example 31: Compound 37

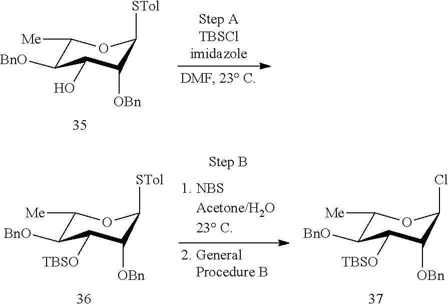

Step A: To a stirred solution of 35 (2.5 g, 5.6 mmol, 1.0 equiv.) in DMF at 23° C. was added imidazole (1.13 g, 16.8 mmol, 3.0 equiv.) followed by TBSCl (1.25 g, 8.3 mmol, 1.5 equiv.). After 12 hours, the reaction was diluted with brine, and extracted three times with ethyl acetate. The combined organic layers were dried over sodium sulfate and concentrated in vacuo. Silica gel chromatography eluting with 5% EtOAc in hexanes furnished compound 36 (2.49 g, 78% yield. TLC: R_{f}=0.50 (hexane/AcOEt=9:1);

¹H NMR (500 MHz, CDCl₃): δ 7.39-7.29 (m, 12H), 7.10 (d, J=8.0 Hz, 2H), 5.40 (s, 1H), 4.93 (d, J=11.0 Hz, 1H), 4.78-4.61 (m, 3H), 4.16-4.13 (m, 1H), 4.08-4.05 (m, 1H), 3.88 (s, 1H), 3.60-3.57 (m, 1H), 2.33 (s, 3H), 1.30 (d, J=6.0 Hz, 3H), 0.98 (s, 9H), 0.21 (s, 6H);

¹³C NMR (126 MHz, CDCl₃): δ 138.58, 138.26, 137.39, 131.85, 131.01, 129.76, 128.30, 128.27, 127.75, 127.67, 127.59, 127.47, 86.63, 81.42, 80.96, 75.35, 73.53, 72.87, 69.47, 25.99, 21.07, 18.06, 17.84, −4.39, −4.66;

IR (cm⁻¹): 3030, 2915, 1496, 1453, 1362, 1208, 1141, 1070, 1026, 911, 841, 788, 733, 694, 655, 612, 545, 484, 458.

Step B: To a stirred solution of 36 (2.49 g, 4.4 mmol, 1.0 equiv.) in 5:1 acetone:water (24 ml) at 23° C. was added NBS (2.36 g, 13.2 mmol, 3.0 equiv.) portion-wise over 10 minutes. At 2 hours the reaction was quenched with saturated aqueous Na₂S₂O₃ and extracted with ethyl acetate. The combined organic layers were washed with brine, dried over sodium sulfate and concentrated in vacuo. The crude material (1.7 g, 3.7 mmol, 1.0 equiv.) was subjected to General Procedure B. Following silica gel plug filtration—10:1 hexanes:ethyl acetate, 37 was obtained (1.5 g), in 85% yield. This compound had limited stability and was used immediately following NMR analysis. TLC: R_{f}=0.50 (hexane/AcOEt=12:1).

¹H NMR (500 MHz, CDCl₃): δ 7.44-7.32 (m, 10H), 6.05 (s, 1H), 5.00-4.88 (m, 2H), 4.73-4.66 (m, 2H), 4.45-4.42 (m, 1H), 4.03-3.99 (m, 1H), 3.85-3.84 (m, 1H), 3.66-3.61 (m, 1H), 1.35 (d, J=6.5 Hz, 3H), 1.03 (s, 9H), 0.19 (s, 6H);

¹³C NMR (126 MHz, CDCl₃): δ 138.24, 137.88, 128.41, 128.26, 128.08, 127.98, 127.84, 127.64, 127.54, 92.08, 82.15, 80.47, 75.37, 73.79, 71.57, 71.33, 25.92, 17.54, −4.49, −4.74.

Alternative Preparation of Compound 37

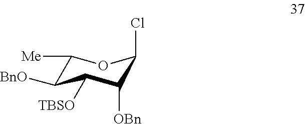

α anomer of 37 was prepared using General Procedure D. Yield: 3.68 mmol, 1.51 g, 91%; TLC: R_{f}=0.60 (hexane/AcOEt=4:1);

¹H NMR (500 MHz, CDCl₃): δ 7.39-7.34 (m, 15H), 6.05 (d, J=1.0 Hz, 1H), 5.00 (d, J=11.0 Hz, 1H), 4.77-4.63 (m, 5H), 4.19 (dd, J=9.5, 3.0 Hz, 1H), 4.02 (p, J=3.0 Hz, 1H), 3.92 (d, J=2.0 Hz, 1H), 3.71 (d, J=9.5 Hz, 1H), 1.39 (d, J=6.5 Hz, 3H);

¹³C NMR (126 MHz, CDCl₃): δ 138.22, 138.05, 137.55, 128.43, 128.37, 128.34, 127.94, 127.88, 127.74, 127.69, 91.58, 79.62, 78.23, 77.98, 75.44, 73.01, 72.40, 71.04, 53.36, 46.10, 17.56.

Characterization matches literature precedent (P. J. Garegg, T. et al., Carbohydrate Res., 1983, 116, 308-311).

### Example 32: Compound 38

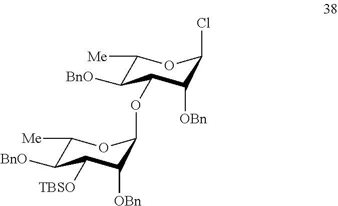

α anomer of 38 was prepared using General Procedure B. Yield: 1.11 mmol, 711 mg, 76%; α:β=1:0; TLC: R_{f}=0.45 (hexane/AcOEt=4:1);

¹H NMR (500 MHz, CDCl₃): δ 7.49 (d, J=7.5 Hz, 2H), 7.39-7.29 (m, 18H), 7.01 (d, J=9.0 Hz, 2H), 6.86 (d, J=9.0 Hz, 2H), 5.46 (d, J=1.5 Hz, 1H), 5.26 (s, 1H), 4.98 (d, J=11.0 Hz, 1H), 4.84-4.66 (m, 6H), 4.45 (d, J=11.0 Hz, 1H), 4.36 (dd, J=9.5, 2.0 Hz, 1H), 4.27 (dd, J=9.5, 2.0 Hz, 1H), 4.00 (t, J=2.0 Hz, 1H), 3.95-3.89 (m, 1H), 3.80 (s, 3H), 3.75-3.73 (m, 1H), 3.61 (t, J=9.0 Hz, 1H), 1.32 (d, J=6.0 Hz, 3H), 1.31 (d, J=6.0 Hz, 3H), 1.00 (s, 9H), −0.15 (s, 3H), −0.14 (s, 3H);

¹³C NMR (126 MHz, CDCl₃): δ 154.78, 150.27, 138.84, 138.62, 138.33, 137.96, 128.46, 128.39, 128.12, 127.70, 127.63, 127.57, 127.30, 127.23, 127.02, 117.50, 114.51, 100.43, 96.56, 81.28, 80.66, 80.00, 78.07, 74.97, 74.85, 73.22, 73.18, 72.86, 69.94, 68.81, 55.56, 25.96, 18.03, 17.93, −4.36, −4.71;

IR (cm⁻¹): 2927, 2854, 1506, 1453, 1386, 1360, 1266, 1249, 1214, 1124, 1095, 1049, 1028, 1003, 927, 870, 835, 796, 776, 732, 695, 611, 520, 459, 427; MS-ESI (m/z): [M+NH₄]⁺ calcd. For [C₅₃H₇₀NO₁₀Si]⁺, 908.4769. found 908.4763.

### Example 33: Compound 39

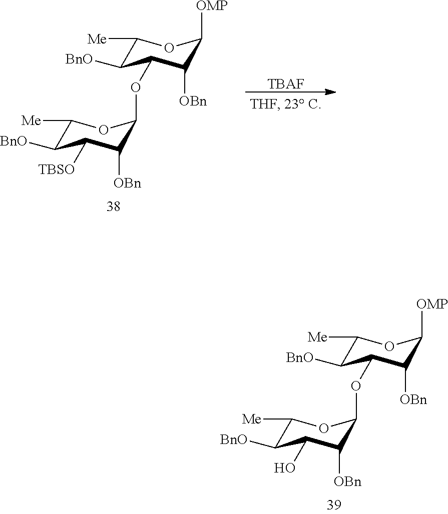

To a stirred solution of 38 (700 mg, 0.79 mmol, 1.0 equiv.) in THF (5 mL) at 23° C. was added a solution of TBAF (1.0 M in THF, 1.58 mL, 2.0 equiv.) dropwise. After 6 h, the reaction was diluted with ethyl acetate and washed with water 2×, and brine 1×. The organic layer was dried over sodium sulfate and concentrated in vacuo. Silica gel chromatography (5:1 hexanes:ethyl acetate) furnished 39 (550 mg) in 92% yield. TLC: R_{f}=0.30 (hexane/AcOEt=4:1).

¹H NMR (500 MHz, CDCl₃): δ 7.46-7.30 (m, 16H), 7.29-7.28 (m, 2H), 7.21 (d, J=7.0 Hz, 2H), 7.01 (d, J=9.0 Hz, 2H), 6.86 (d, J=9.0 Hz, 2H), 5.43 (s, 1H), 5.32 (s, 1H, H-1), 4.97 (d, J=11.0 Hz, 1H), 4.87-4.78 (m, 4H), 4.70 (d, J=11.0 Hz, 1H), 4.42-4.36 (m, 2H), 4.20-4.18 (m, 1H), 4.08 (bs, 1H), 3.96-3.88 (m, 3H), 3.80 (s, 3H), 3.77-3.73 (m, 2H), 3.40 (t, J=9.5 Hz, 1H), 1.36 (d, J=6.0 Hz, 3H), 1.35 (d, J=6.0 Hz, 3H);

¹³C NMR (126 MHz, CDCl₃): δ 154.78, 150.21, 138.68, 138.37, 137.77, 137.69, 128.42, 128.37, 128.22, 128.23, 127.71, 127.67, 127.53, 127.48, 126.74, 117.50, 114.50, 98.79, 96.66, 82.11, 80.83, 79.04, 77.61, 77.47, 74.77, 72.84, 72.41, 71.56, 68.90, 67.79, 55.53, 17.99, 17.94;

IR (cm⁻¹): 2932, 1735, 1506, 1453, 1372, 1215, 1135, 1093, 1027, 920, 827, 733, 696, 634, 607, 518, 459, 436; MS-ESI (m/z): [M+NH₄]⁺ calcd. For [C₄₇H₅₆NO₁₀]⁺, 794.3904. found 794.3895.

### Example 34: Compound 40

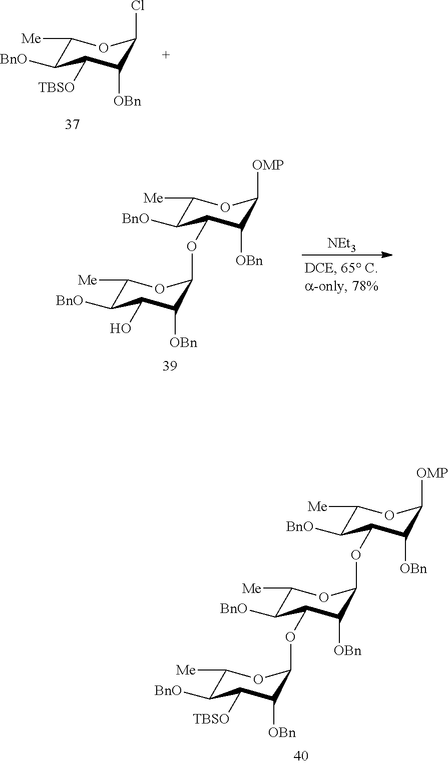

α anomer of 40 was prepared by using General Procedure B. Yield: 0.64 mmol, 305 mg, 78%; α:β=1:0; TLC: R_{f}=0.30 (hexane/AcOEt=4:1).

¹H NMR (500 MHz, CDCl₃): δ 7.47 (d, J=7.0 Hz, 2H), 7.37-725 (m, 28H), 6.98 (d, J=9.5 Hz, 2H), 6.84 (d, J=9.5 Hz, 2H), 5.43 (d, J=1.5 Hz, 1H), 5.28 (s, 1H), 5.16 (d, J=1.0 Hz, 1H), 4.93 (d, J=11.5 Hz, 1H), 4.82-4.61 (m, 8H), 4.44 (s, 2H), 4.40-4.37 (d, J=12.0 Hz, 1H), 4.33 (dd, J=9.5, 3.0 Hz, 1H), 4.23 (dd, J=9.5, 3.0 Hz, 1H), 4.20 (dd, J=9.5, 3.0 Hz, 1H), 3.96 (t, J=2.0 Hz, 1H), 3.90-3.83 (m, 4H), 3.79 (s, 3H), 3.73-3.63 (m, 3H), 3.55 (t, J=9.0 Hz, 1H), 1.28 (d, J=6.5 Hz, 6H), 1.20 (d, J=6.5 Hz, 3H), 0.95 (s, 9H), 0.09 (s, 3H), 0.08 (s, 3H);

¹³C NMR (126 MHz, CDCl₃): δ 154.81, 150.28, 138.89, 138.65, 138.62, 138.30, 138.13, 137.89, 128.49, 128.40, 128.31, 128.12, 128.09, 127.74, 127.72, 127.59, 127.43, 127.31, 127.23, 127.19, 127.07, 126.83, 117.52, 114.53, 100.51, 99.62, 96.50, 81.28, 80.66, 80.58, 80.01, 78.93, 78.56, 78.19, 77.94, 74.88, 74.63, 73.24, 72.84, 72.35, 68.87, 68.4, 68.73, 55.60, 25.96, 18.12, 17.97, 17.93, 18.0, −4.40, −4.73;

IR (cm⁻¹): 2928, 2856, 1508, 1497, 1453, 1386, 1360, 1288, 1252, 1213, 1094, 1041, 1027, 921, 868, 835, 801, 776, 732, 695, 613, 562, 518, 489, 478, 459; MS-ESI (m/z): [M+NH₄]⁺ calcd. For [C₇₃H₉₂NO₁₄Si]⁺, 1234.6287. found 1234.6280.

### Example 35: Compound 41

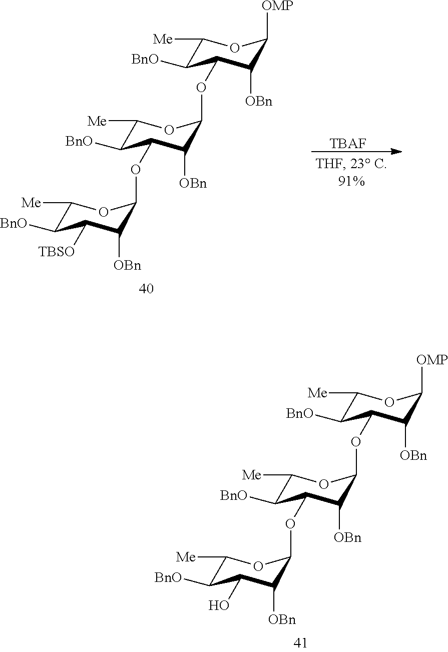

To a stirred solution of 40 (230 mg, 0.19 mmol, 1.0 equiv.) in THF (3 mL) at 23° C. was added a solution of TBAF (1.0 M THF, 0.38 mL, 0.38 mmol, 2.0 equiv.) dropwise. After 6 h, the reaction was diluted with ethyl acetate and washed with water 2×, and brine 1×. The organic layer was dried over sodium sulfate and concentrated in vacuo. Silica gel chromatography (7:1 hexanes:ethyl acetate) furnished 41 (190 mg) in 91% yield. TLC: R_{f}=0.25 (hexane/AcOEt=4:1).

¹H NMR (500 MHz, CDCl₃): δ 7.45 (d, J=7.5 Hz, 2H), 7.35-7.18 (m, 26H), 7.13 (d, J=6.5 Hz, 2H), 6.95 (d, J=9.5 Hz, 2H), 6.81 (d, J=9.5 Hz, 2H), 5.40 (d, J=1.5 Hz, 2H), 5.22 (s, 1H), 5.19 (s, 1H), 4.87 (d, J=12.0 Hz, 1H), 4.82-60 (m, 7H), 4.44-4.42 (m, 2H), 4.39-4.27 (m, 2H), 4.22 (dd, J=10.0, 3.0, Hz, 1H), 4.10-4.08 (m, 1H), 3.98-3.78 (m, 5H), 3.77-3.75 (m, 4H), 3.69-3.61 (m, 3H), 3.31 (t, J=9.0 Hz, 1H), 2.25 (d, J=9.5 Hz, 1H), 1.28 (d, J=6.5 Hz, 3H), 1.24 (d, J=6.5 Hz, 3H), 1.20 (d, J=6.5 Hz, 3H);

¹³C NMR (126 MHz, CDCl₃): δ 154.82, 150.30, 138.75, 138.65, 138.24, 137.98, 137.76, 128.49, 128.42, 128.36, 128.29, 128.26, 127.75, 127.66, 127.50, 127.33, 127.12, 126.61, 117.54, 114.55, 99.70, 98.85, 96.44, 82.17, 80.89, 80.57, 79.12, 78.54, 78.26, 78.07, 74.89, 74.69, 74.48, 72.82, 72.45, 72.36, 71.51, 68.83, 67.71, 55.61, 18.13, 18.03, 17.94;

IR (cm⁻¹): 2917, 1506, 1497, 1453, 1386, 1360, 1288, 1252, 1213, 1094, 1041, 1027, 921, 868, 835, 801, 776, 732, 695, 613, 562, 518, 489, 478, 459; MS-ESI (m/z): [M+NH₄]⁺ calcd. For [C₆₇H₇₈NO₁₄]⁺, 1120.5422. found 1120.5413.

### Example 36: Compound 42

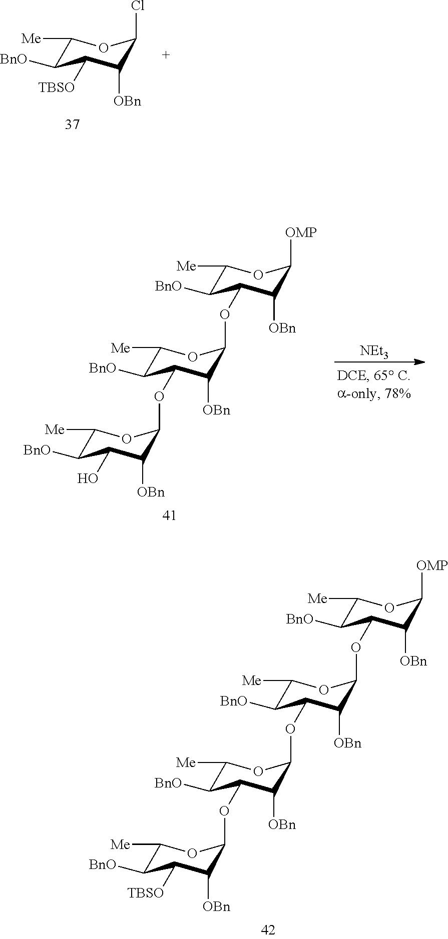

α anomer of 42 was prepared using General Procedure B. Yield: 0.06 mmol, 70 mg, 72%; α:β=1:0; TLC: R_{f}=0.25 (hexane/AcOEt=4:1);

¹H NMR (500 MHz, CDCl₃): δ 7.45 (d, J=7.5 Hz, 2H), 7.35-7.20 (m, 38H), 6.96 (d, J=9.5 Hz, 2H), 6.82 (d, J=9.5 Hz, 2H), 5.40 (s, 1H), 5.25 (s, 1H), 5.18 (s, 1H), 5.11 (s, 1H), 4.90 (d, J=12.0 Hz, 1H), 4.82-4.55 (m, 9H), 4.48-4.45 (m, 1H), 4.39-4.29 (m, 5H), 4.20 (dd, J=9.5, 3.0 Hz, 1H), 4.17-4.13 (m, 2H), 3.94 (bs, 1H), 3.88-3.84 (m, 3H), 3.81-3.76 (m, 5H), 3.70-3.59 (m, 4H), 3.50 (t, J=9.0, 1H), 1.27 (d, J=6.5 Hz, 3H), 1.26 (d, J=6.5 Hz, 3H), 1.15 (d, J=6.5 Hz, 6H);

¹³C NMR (126 MHz, CDCl₃): δ 154.82, 150.31, 138.92, 138.67, 138.57, 138.30, 138.18, 138.05, 137.92, 128.49, 128.49, 128.42, 128.32, 128.28, 128.11, 128.09, 127.75, 127.72, 127.62, 127.46, 127.40, 127.34, 127.29, 127.25, 127.19, 127.06, 126.88, 126.76, 117.54, 114.55, 100.44, 99.75, 99.54, 96.46, 81.29, 80.70, 80.57, 80.03, 78.95, 78.80, 78.67, 78.47, 78.22, 78.02, 74.88, 74.66, 74.52, 73.19, 73.14, 72.85, 72.36, 72.30, 68.84, 68.81, 68.61, 55.62, 25.96, 18.10, 18.02, 17.98, 17.95, −4.40, −4.73;

IR (cm⁻¹): 3030, 2920, 1726, 1702, 1493, 1453, 1365, 1314, 1274, 1206, 1085, 1026, 910, 844, 808, 795, 733, 695, 616, 485, 462, 426;

HRMS-ESI (m/z): [M+NH₄]⁺ calcd. For [C₉₃H₁₁₄NO₁₈Si]⁺, 1560.7805. found 1560.7800.

### Example 37—Compound 43

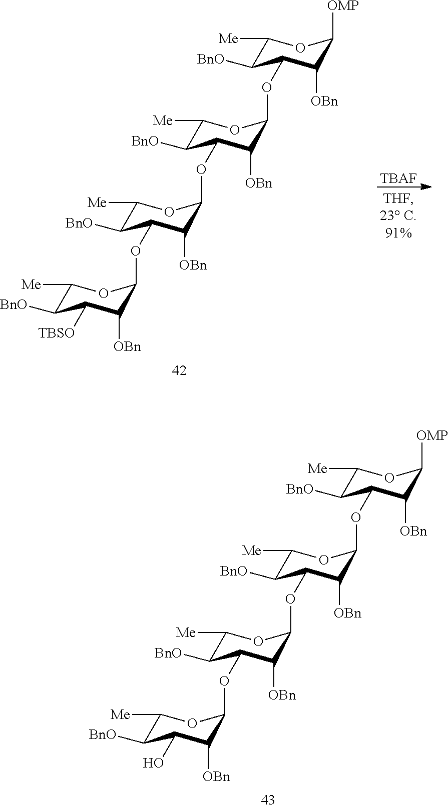

To a stirred solution of 42 (65 mg, 0.04 mmol, 1.0 equiv.) in THF (0.4 mL) at 23° C. was added a solution of TBAF (1.0 M THF, 0.08 mL, 0.08 mmol, 2.0 equiv.) dropwise. After 6 h, the reaction was diluted with ethyl acetate and washed with water 2×, and brine 1×. The organic layer was dried over sodium sulfate and concentrated in vacuo. Silica gel chromatography (5:1 hexanes:ethyl acetate) furnished 43 (54 mg) in 95% yield. TLC: R_{f}=0.60 (hexane/AcOEt=4:1)

¹H NMR (500 MHz, CDCl₃): δ 7.44 (d, J=7.5 Hz, 2H), 7.34-7.21 (m, 36H), 7.12 (d, J=6.0 Hz, 2H), 6.96 (d, J=9.0 Hz, 2H), 6.81 (d, J=9.0 Hz, 2H), 5.40 (d, J=1.5 Hz, 1H), 5.26 (s, 1H), 5.17 (s, 1H), 5.16 (s, 1H), 4.87 (d, J=11.0 Hz, 1H), 4.81-4.60 (m, 7H), 4.47-4.45 (m, 1H), 4.39-4.27 (m, 5H,), 4.19 (dd, J=9.5, 2.5 Hz, 2H), 4.07-4.04 (m, 1H), 3.97-3.93 (m, 2H), 3.87-3.3.80 (m, 4H), 3.79-3.3.70 (m, 5H), 3.67-3.60 (m, 4H), 3.28 (t, J=9.0 Hz, 1H), 2.25 (d, J=9.5 Hz, 1H), 1.26 (d, J=6.5 Hz, 3H), 1.25 (d, J=6.5 Hz, 3H), 1.18 (d, J=6.5 Hz, 3H), 1.17 (d, J=6.5 Hz, 3H);

¹³C NMR (126 MHz, CDCl₃): δ 154.82, 150.30, 138.76, 138.67, 138.50, 138.33, 138.10, 138.00, 137.90, 137.77, 128.50, 128.42, 128.33, 128.27, 127.77, 127.71, 127.66, 127.49, 127.32, 127.30, 127.02, 126.93, 126.53, 117.54, 114.55, 99.83, 99.45, 98.79, 96.46, 82.18, 80.91, 80.59, 79.12, 78.92, 78.57, 78.20, 78.02, 77.80, 74.86, 74.69, 74.37, 72.86, 72.39, 72.24, 71.50, 68.85, 68.73, 67.68, 60.37 55.63, 18.10, 17.99, 17.95;

IR (cm⁻¹): 2931, 1506, 1453, 1386, 1283, 1214, 1094, 1040, 1027, 918, 827, 733, 695, 606, 519, 459;

HRMS-ESI (m/z): [M+NH₄]⁺ calcd. For [C₈₇H₁₀₀NO₁₈]⁺, 1446.6940. found 1446.6934.

### Example 38: Compound 44

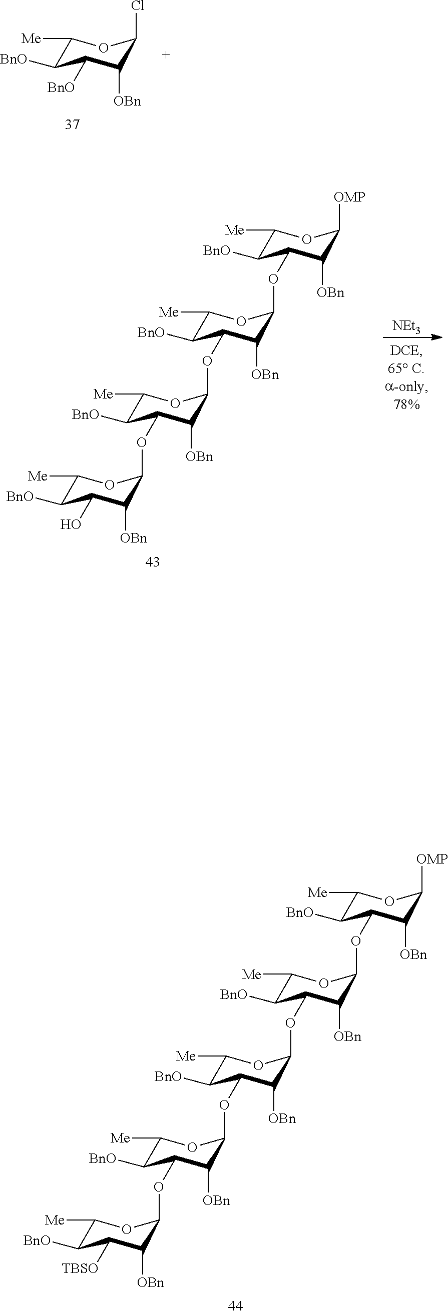

α anomer of 44 was prepared using General Procedure B. Yield: 0.036 mmol, 57 mg, 86%, α:β=1:0; R_{f}=0.30 (hexane/AcOEt=4:1);

¹H NMR (500 MHz, CDCl₃): δ 7.46 (d, J=7.5 Hz, 2H), 7.36-7.18 (m, 53H), 6.97 (d, J=9.0 Hz, 2H), 6.83 (d, J=9.0 Hz, 2H), 5.41 (d, J=1.5 Hz, 1H), 5.26 (s, 1H), 5.18 (s, 1H), 5.14 (s, 1H), 5.13 (s, 1H), 4.95 (d, J=11.0 Hz, 1H), 4.80-4.74 (m, 4H), 4.72-4.59 (m, 6H), 4.56-4.50 (m, 3H), 4.47-4.42 (m, 4H), 4.41-4.30 (m, 4H), 4.21 (dd, J=9.5, 3.0 Hz, 1H), 4.17-4.13 (m, 2H), 3.95 (t, J=2.5 Hz, 1H), 3.88-3.86 (m, 4H), 3.85-3.76 (m, 9H), 3.73-3.59 (m, 4H), 3.57-3.51 (m, 1H), 1.27 (d, J=6.5 Hz, 6H, 2*CH₃), 1.20 (d, J=6.5 Hz, 3H), 1.16 (d, J=6.5 Hz, 3H), 1.12 (d, J=6.5 Hz, 3H);

¹³C NMR (126 MHz, CDCl₃): δ 154.82, 150.29, 138.92, 138.63, 138.54, 138.34, 138.30, 138.13, 138.06, 137.90, 128.49, 128.41, 128.30, 128.25, 128.21, 128.18, 128.14, 127.76, 127.70, 127.63, 127.56, 127.46, 127.37, 127.31, 127.27, 127.03, 126.84, 126.72, 117.53, 114.54, 99.64, 99.47, 96.45, 80.78, 80.62, 80.57, 80.43, 79.77, 78.83, 78.60, 78.22, 78.04, 75.70, 74.97, 74.86, 74.64, 74.56, 74.37, 72.85, 72.43, 72.27, 72.04, 68.85, 68.74, 68.65, 68.59, 55.62, 18.14, 18.03, 17.99, 17.95;

IR (cm⁻¹): 2930, 1496, 1453, 1387, 1356, 1282, 1213, 1175, 1097, 1041, 1027, 1005, 919, 828, 731, 694, 611, 540, 477, 456, 432;

HRMS-ESI (m/z): [M+NH₄]⁺ calcd. For [C₁₁₄H₁₂₈NO₂₂]⁺, 1862.8923. found 1862.8911.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. The terms “including” and “having” are defined as comprising (i.e., open language).

Those skilled in the art will recognize that numerous modifications can be made to the specific implementations described above. The implementations should not be limited to the particular limitations described. Other implementations may be possible. While the inventions have been illustrated and described in detail in the drawings and foregoing description, the same is to be considered illustrative and not restrictive in character, it being understood that only certain embodiments have been shown and described and that all changes and modifications that come within the spirit of the invention are desired to be protected.

We claim:

1. A catalytic stereoselective glycosylation method for preparing an α-1,2-cis glycosidic linkage, which method comprises:
reacting a glycosyl donor of formula (I): 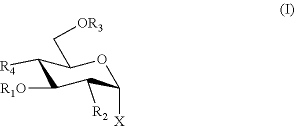
wherein
each of R₁ and R₃ is independently a substituted or an unsubstituted group selected from alkyl, heteroalkyl, cycloalkyl, heterocycloalkyl, aryl, heteroaryl, alkynyl, alkenyl, heteroarylalkyl, heteroalkylaryl, arylalkenyl, allyl, arylalkyl, alkylcarboxyl, arylcarboxyl, trialkylsilyl, diarylalkylsilyl, dialkylarylsilyl, alkoxyalkyl, tetrahydropyranyl, benzyl, and benzoyl, wherein each substituted group can be independently substituted with at least one of alkyl, aryl, heteroaryl, halo, cyano, alkoxy, amino, carboxyl, amide, and ester;
each of R₂ and R₄ is independently alkyl, N₃ or OR₁, wherein R₁ is as defined above; or
any two adjacent groups selected from OR₁ and R₂, OR₁ and R₄, and OR₃ and R₄ are linked together to form a 5- to 7-membered cyclic ring, wherein the cyclic ring can be optionally independently substituted with at least one of alkyl, aryl, heteroaryl, halo, cyano, alkoxy, trialkylsilyl, diarylalkylsilyl, amino, carboxyl, amide, and ester group; and
X is a halide selected from Cl, Br, I and F;
with a glycosyl acceptor in the presence of a base and an isothiourea catalyst of formula (II): 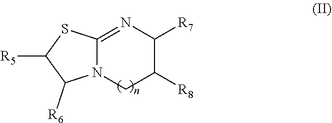
wherein n is 0 to 3;
each of R₅ and R₆ is H, COOR₉, CONHR₉, CONR₉R₉, COR₉, or a substituted or an unsubstituted group selected from alkyl, heteroalkyl, cycloalkyl, heterocycloalkyl, aryl, heteroaryl, alkynyl, alkenyl, heteroarylalkyl, heteroalkylaryl, arylalkenyl, and arylalkyl, wherein R₉ is alkyl or aryl;
each of R₇ and R₅ is H, nitrile, nitro, OR₉, COOR₉, CONHR₉, CONR₉R₉, COR₉, SO₂R₉ or a substituted or an unsubstituted group selected from alkyl, heteroalkyl, cycloalkyl, heterocycloalkyl, aryl, heteroaryl, alkynyl, alkenyl, heteroarylalkyl, heteroalkylaryl, arylalkenyl, and arylalkyl, wherein R₉ is alkyl or aryl; or
R₅ and R₆ and/or R₇ and R₈ are linked together to form an aliphatic, an aromatic, or a heterocyclic ring, each of which is optionally independently substituted with a group selected from alkyl, aryl, heteroaryl, halo, cyano, alkoxy, amino, carboxyl, amide and ester.

2. The method of claim 1, wherein the glycosyl donor is a glycosyl halide selected from: 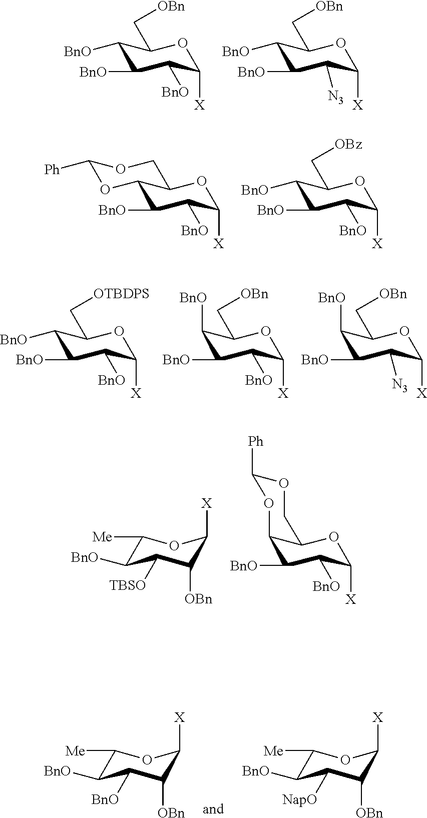
wherein X is Cl, Br, I and F.

3. The method of claim 1, wherein the isothiourea catalyst is:

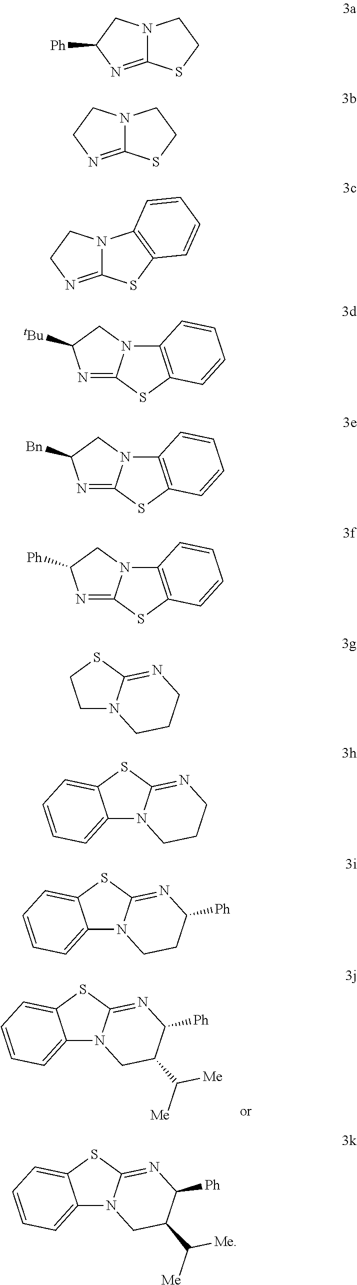

4. The method of claim 1, wherein the glycosyl acceptor comprises an alcohol, a thiol, or an amine.

5. The method of claim 4, wherein the glycosyl acceptor is a monosaccharide, a disaccharide, an oligosaccharide, or a polysaccharide, each comprising at least one —OH, —SH, or a primary or secondary amino group.

6. The method of claim 5, wherein the glycosyl acceptor is selected from:

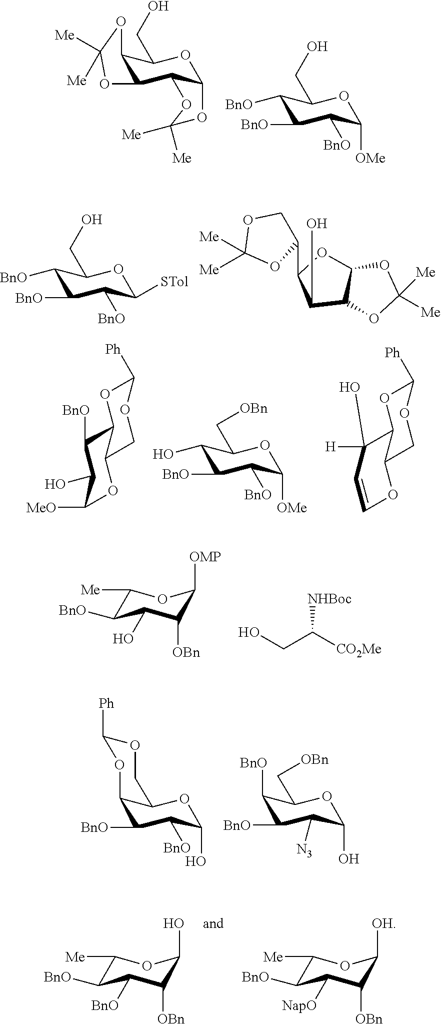

7. The method of claim 1, wherein the base is selected from triethylamine, tri-tert-butylpyrimidine (TTBP), 2,6-di-tert-butylpyridine, N,N-diisopropyl ethylamine (DIPEA), 1,8-diazabicycloundec-7-ene (DBU), 2,6-lutidine, and 2,4,6-collidine.

8. The method of claim 7, wherein the base is triethylamine.

9. The method of claim 1, wherein the α-1,2-cis glycosidic linkage is formed with about 80% to about 99% stereoselectivity.

10. The method of claim 9, wherein the α-1,2-cis glycosidic linkage is formed with greater than or equal to 95% stereoselectivity.

11. The method of claim 9, wherein the α-1,2-cis glycosidic linkage is formed with greater than or equal to 98% stereoselectivity.

12. The method of claim 9, wherein the α-1,2-cis glycosidic linkage is formed with greater than or equal to 99% stereoselectivity.

13. The method of claim 1, wherein the method further comprises the use of an organic solvent.

14. The method of claim 13, wherein the organic solvent is selected from dichloromethane, dichloroethane, chloroform, ethyl acetate, toluene, tetrahydrofuran, dimethylformamide, acetone, benzene, and methyl-tert-butyl ether.

15. The method of claim 14, wherein the organic solvent is dichloroethane.

16. The method of claim 1, wherein the catalytic stereoselective glycosylation is carried out at a temperature from about room temperature to about 100° C.

17. The method of claim 16, wherein the catalytic stereoselective glycosylation is carried out at a temperature of about 65° C.

18. The method of any one of claim 1, wherein the α-1,2-cis glycosidic linkage forms a compound selected from: 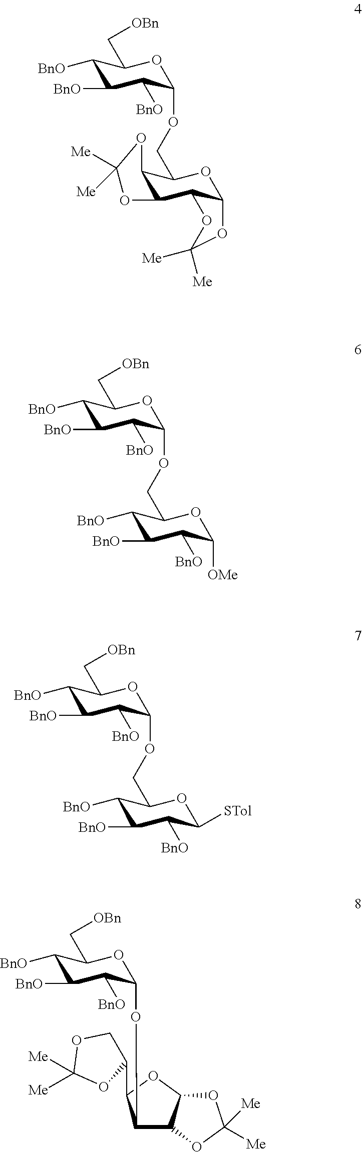 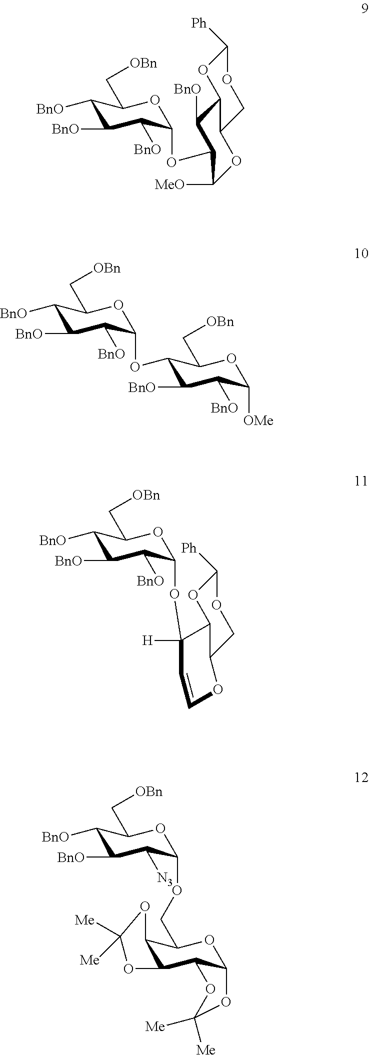 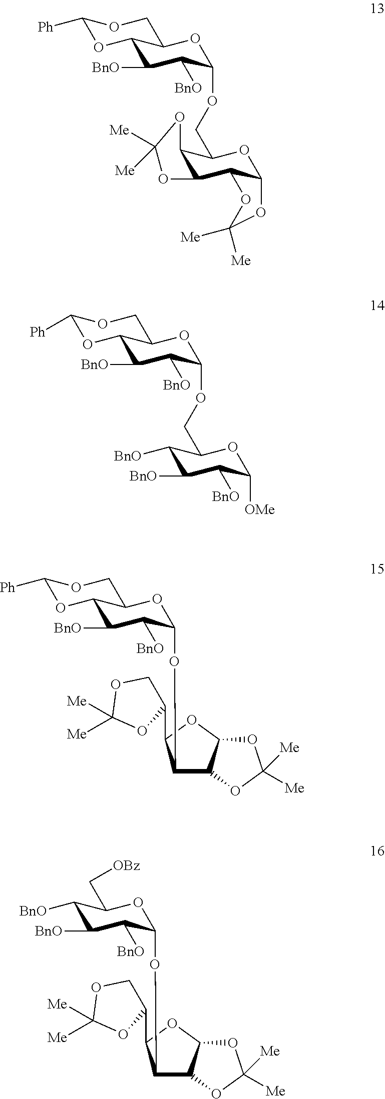 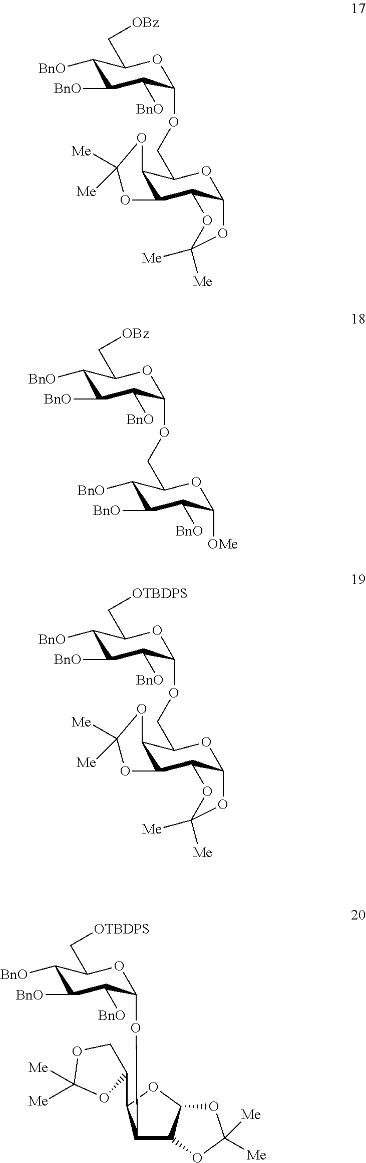 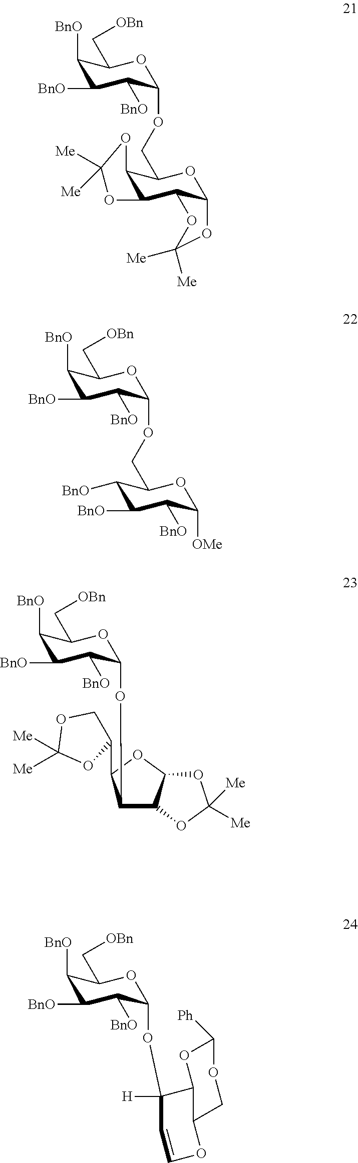 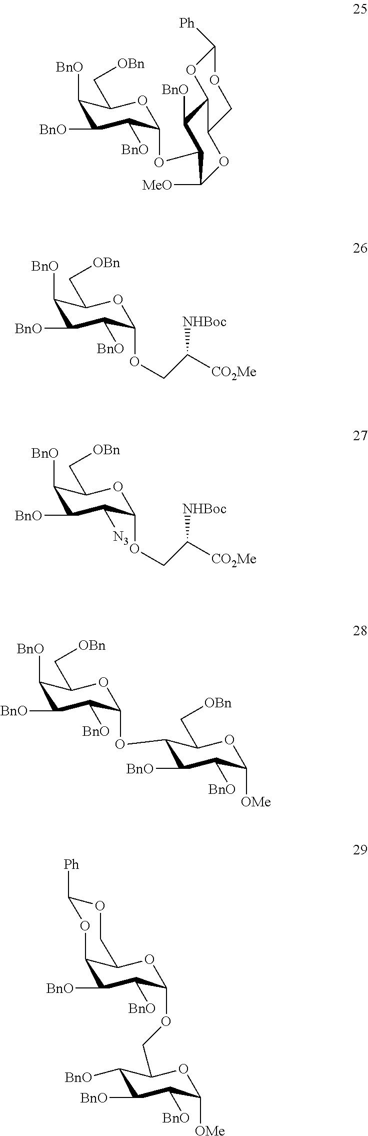 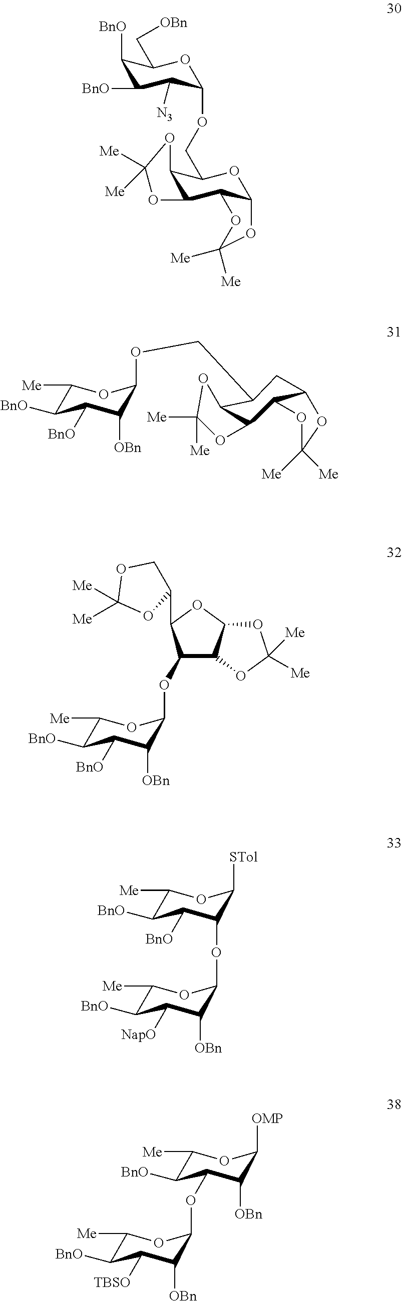 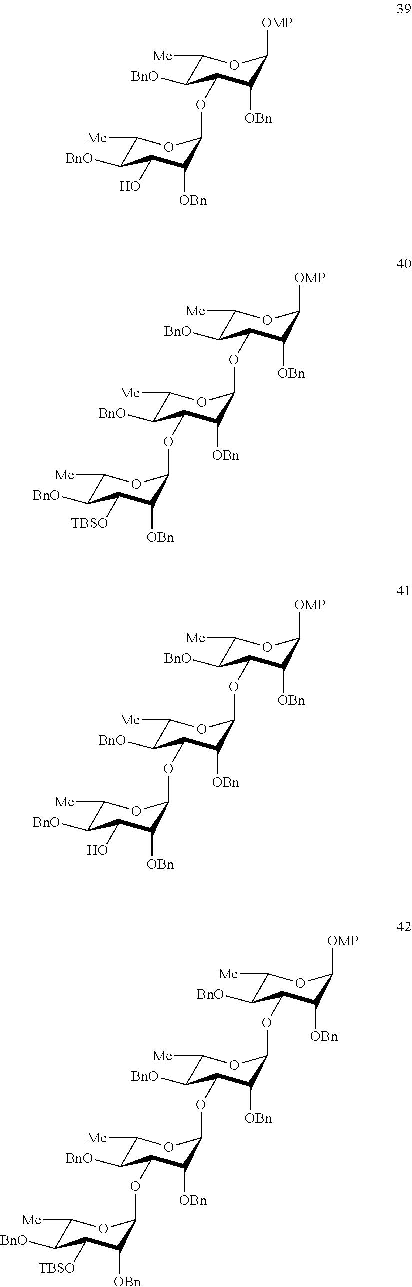 [chemical structure]
